(19) United States
(12) Patent Application Publication
(10) Pub. No.: US 2024/0043892 A1
(43) Pub. Date: Feb. 8, 2024

(54) METHODS FOR IN VITRO TRANSCRIPTION

(71) Applicant: Arcturus Therapeutics, Inc., San Diego, CA (US)

(72) Inventors: Maher ALAYYOUBI, San Diego, CA (US); Kyoung-Joo Jenny CHOI, San Diego, CA (US); Timothy Wayne LUGER, II, San Diego, CA (US)

(21) Appl. No.: 18/345,379

(22) Filed: Jun. 30, 2023

Related U.S. Application Data
(60) Provisional application No. 63/357,843, filed on Jul. 1, 2022.

(51) Int. Cl. C12P 19/34; C12N 9/10; C12N 9/12

(52) U.S. Cl. CPC C12P 19/34; C12N 9/1007; C12Y 201/01; C12N 9/1247; C12Y 207/07006

(57) ABSTRACT

Disclosed herein are methods of producing transcribed RNA product with increased yield and reduced dsRNA impurities.

### CROSS-REFERENCE TO RELATED APPLICATIONS

This application claims priority to U.S. Provisional Application No. 63/357,843, filed Jul. 1, 2022, which is incorporated herein by reference in its entirety.

### FIELD

The present application generally relates to methods for in vitro transcription.

### BACKGROUND

RNA-based therapeutics and vaccines are at the frontier of modern medicine providing hope for those suffering from genetic diseases and those fearing deadly pathogens. For example, messenger RNA (mRNA) can be used in protein replacement therapy to treat diseases caused by a lack of protein, or by defective proteins, such as in cystic fibrosis. If a gene has a mutation that stops it from producing protein or causes it to produce defective protein, mRNA medicine can provide a healthy version of the missing protein.

RNA-based vaccines have emerged as a new class of RNA medicines. RNA vaccines can be developed more rapidly than traditional vaccines in response to infectious disease outbreaks as shown by the first two vaccines to obtain emergency use authorization from the FDA for the prevention of COVID-19, a deadly viral infection caused by SARS-CoV-2.

One challenge to the development of RNA-based therapeutics and vaccines is the robust and efficient manufacture of mRNA with high yields and low levels of impurities, such as double-stranded RNA (dsRNA). Double-stranded RNA is an aberrant by-product of the in vitro transcription (IVT) enzymatic reaction. It induces the immune response, inhibits protein translation, and hence decreases the safety/efficacy of the mRNA therapeutics and vaccines. Hence, there is an urgent need in manufacturing processes to address the removal of dsRNA either at the IVT level or in downstream purification steps. Another problem in IVT manufacturing that remains unaddressed is how to increase the yield of mRNA per given IVT volume. In manufacturing, there are many challenges associated with materials (e.g., costs, limited supplies), capacity conforming to good manufacturing practices (GMP) (e.g., limited slots available, significant time and labor required for preparation of a single run), and equipment (e.g., availability, validation, handling). Maximizing the amount of mRNA manufactured at a given scale leads to reduction in material cost, number of expensive GMP runs, time, and labor, and even in some cases, eliminates the need of validating larger-scale equipment and processes. Thus, there is an urgent need to develop mRNA manufacturing processes that reliably and efficiently produce mRNA with increased yields and with reduced dsRNA.

### SUMMARY

Described herein are methods of producing transcribed RNA product with increased yield and reduced dsRNA impurities.

Accordingly, the present application relates to the result that the combination of increased nucleoside triphosphates (NTPs), Mg²⁺, and time led to high yields (measured in g/L) of mRNA and that such conditions resulted in much lower levels of double-stranded RNA (dsRNA) a common impurity in in vitro transcription (IVT) reactions.

In another aspect, the present application also relates to the surprising result that the addition of organic solvents can reduce dsRNA by over an order of magnitude.

In another aspect, the present application also relates to the surprising result that increasing the salt concentration in the linear DNA (L.DNA) stock prior to the IVT reaction leads to reduced dsRNA without inhibiting yield in IVT reactions using wild-type and mixed-wild-type NTPs.

In an aspect, the present application provides a method of producing a transcribed RNA product comprising:
(a) reacting a transcription reaction mixture comprising a buffer solution comprising Mg²⁺, linear DNA (L.DNA) template, ribonucleoside tri-phosphate (rNTPs), optionally an RNA capping reagent, and RNA polymerase; wherein a molar concentration of Mg²⁺ is 2 to 15 mM above the total molar concentration of all rNTPs plus the optional RNA capping reagent; and a RNA polymerase/L.DNA template mass ratio is between 0.25 and 3;
(b) stopping the transcription reaction by digesting the L.DNA template with deoxyribonuclease (DNase) or quenching the RNA polymerase with EDTA;
wherein a yield of about 1 g to about 25 g of single stranded transcribed RNA per liter of solution of the transcription reaction prior to the addition of DNase in step (b) is produced.

Accordingly, it is an object of the invention not to encompass within the invention any previously known product, process of making the product, or method of using the product such that Applicants reserve the right and hereby disclose a disclaimer of any previously known product, process, or method.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 illustrates the yield time course for targeted 5 g/L IVT reaction. The yield plateau occurs at 50 minutes. Post-50 minutes, there was no increase in yield.

FIGS. 2A-2B illustrate time-course studies. (2A) The time-course study showing the yield per incubation time for 10 g/L and (2B) the time-course study showing the yield per incubation time for 15 g/L IVT reaction each performed at 50 ml IVT scale down model.

FIGS. 3A-3B illustrate the increase in yield was accompanied by a decrease in dsRNA level. Increasing the IVT yield to 10 and 15 g/L target yield results in decrease in dsRNA level at (3A) small scale (200 μL) and (3B) 50 mL-scale IVT volumes.

FIGS. 4A-4B illustrate the addition of ethanol and acetonitrile to IVT results. (4A) Addition of ethanol and acetonitrile results in significant decrease of dsRNA levels (compare lane 2 and 3 to lane 1). The ethanol and acetonitrile effect on dsRNA was significant independent of the initial level of dsRNA or IVT condition (compare lane 2 and 3 to 1 and lanes 5 and 6 to 4). (4B) Addition of ethanol and acetonitrile decreases IVT levels of dsRNA regardless of UTP chemistry (i.e., the uridine triphosphate (UTP) or modified UTP used in the IVT reaction) or size of the encoded mRNA (compare lanes 3 to 2 and 5 to 4).

FIG. 5 illustrates ELISA quantitation of the dsRNA levels of the new 10 g/L IVT plus ethanol compared to the 5 g/L condition described in Example 2.

FIGS. 6A-6B illustrate the effect of (6A) isopropyl alcohol (IPA) and (6B) methanol as an IVT additive on the dsRNA level of the IVT product.

FIG. 7 illustrates that increasing the % v/v of the additives results in decrease in IVT yields.

FIG. 8 illustrates the dose-dependent effect of L.DNA salt spike on IVT dsRNA levels of a replicon RNA transcript (aka “replicon”). The results show that the effects were more pronounced for replicon with natural UTP than for the mRNAs with N1 modified UTP. The effect of salt spiking did not affect the yield or purity of the mRNA product until levels above about 1200 mM of NaCl were added. As illustrated in the results, the dsRNA levels decreased as the level of salt spike increased from 200 mM to 1000 mM NaCl.

FIGS. 9A-9D illustrate six different IVT conditions comparing experimental conditions with reference conditions. 9A shows the Mg²⁺ concentration to NTP concentration ratio for each condition. 9B shows the yield for each condition at two different lengths of time (2 hours and 4 hours). 9C shows % full-length transcripts from each condition. 9D shows a dot analysis for each condition, where the lane for condition 4 is not available due to the reaction failing.

FIGS. 10A-10D illustrate reaction conditions 3, 5, and new comparative condition 7. 10A shows the Mg²⁺ concentration to NTP concentration ratio for each condition. 10B shows the yield for each condition at two different lengths of time (2 hours and 4 hours). 10C shows % full-length transcripts from each condition. 10D shows a dot analysis for each condition.

FIGS. 11A-11D illustrate various iterations of reaction conditions 3 and 7. 11A shows the Mg²⁺ concentration to NTP concentration ratio for each condition. 11B shows the yield for each condition at two different lengths of time (2 hours and 4 hours). 11C shows % full-length transcripts from each condition. 11D shows a dot analysis for each condition at 2.5 hours and 4 hours.

FIG. 12 illustrates the effect of guanidine hydrochloride as an IVT additive on the dsRNA level of the IVT product.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, figures and detailed description are to be regarded as illustrative in nature and not as restrictive.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

The term “isolated,” “isolating,” “purified,” and the like, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A nucleic acid that is the predominant species present in a preparation is substantially purified.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, the term “expression” is used in accordance with its plain ordinary meaning and refers to any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “transcription” as used herein, generally refers to the process of copying a segment of DNA into RNA, where the segments of DNA transcribed into RNA molecules that encode proteins produce mRNA. In embodiments, the segments of DNA that are copied into RNA molecules are referred to as non-coding RNAs.

As used herein, the terms “inhibitor,” “repressor” or “antagonist” or “downregulator” are used in accordance with its plain ordinary meaning and refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In instances, expression or activity is 1.5-fold, 2-fold, 3-fold, 4-fold, 5-fold, 10-fold or lower than the expression or activity in the absence of the antagonist.

The term “polymerase” as used herein, generally refers to an enzyme that catalyzes the synthesis of DNA or RNA whose sequence is complementary to the original template. In embodiments, RNA polymerase is the enzyme that synthesizes RNA from a DNA template.

The term “template” as used herein, generally refers to the antisense DNA strand. In embodiments, the cell uses the antisense strand as a template for producing messenger RNA (mRNA) that directs the synthesis of a protein. In embodiments, the term “linear DNA (L.DNA) template” generally refers to DNA antisense strands that have been uncoiled or linearized by the use of restriction enzyme or are PCR amplicons.

The term “DNase” as used herein, generally refers to deoxyribonuclease, which is an enzyme that catalyzes the hydrolytic cleavage of phosphodiester bonds that connect/link nucleotides in the DNA backbone.

As used herein, “transcribed RNA product,” “in vitro transcribed RNA,” “in vitro-synthesized RNA” and the like generally refers to mRNA that is synthesized or prepared using a method comprising in vitro transcription of one or more DNA templates by an RNA polymerase. In embodiments, the in vitro-synthesized RNA encodes (or exhibits a coding sequence of) at least one protein or polypeptide. In some embodiments, the RNA encodes at least one protein that is capable of effecting a biological or biochemical effect when repeatedly or continuously introduced into a human or animal cell (e.g., a mammalian cell). In some embodiments, the disclosure comprises an RNA composition comprising or consisting of in vitro-synthesized RNA that encodes one protein or polypeptide. In embodiments, the disclosure comprises an RNA composition comprising or consisting of a mixture of multiple different in vitro-synthesized ssRNAs or mRNAs, each of which encodes a different protein. Other embodiments of the disclosure comprise an RNA composition comprising or consisting of in vitro-synthesized ssRNA that does not encode a protein or polypeptide, but instead exhibits the sequence of at least one long non-coding RNA (ncRNA). Still other embodiments comprise various reaction mixtures, kits and methods that comprise or use an RNA composition.

The terms “substantially free of dsRNA,” “virtually free of dsRNA,” “essentially free of dsRNA,” “practically free of dsRNA,” “extremely free of dsRNA,” or “absolutely free of dsRNA,” as used herein, respectively, that less than about: 0.5%, 0.1%, 0.05%, 0.01%, 0.001% or 0.0002% of the mass of the RNA in the treated ssRNA composition is dsRNA of a size greater than about 40 basepairs.

In embodiments, the one or more in vitro transcribed RNAs are substantially free of uncapped RNAs that exhibit a 5′-triphosphate group (which are considered to be one type of “contaminant RNA molecules” herein). As used herein, the RNA product is at least 50%, 60,%, 70%, 80%, 90%, 91%, 92%, 93%, 94%, 95%, 96%, 97%, 98%, 99%, 99.5%, or 99.99% free of uncapped RNAs that exhibit a 5′-triphosphate group.

As used herein, the term “salt spiking” refers to the addition of sodium chloride (NaCl) or an equivalent salt to the L.DNA prior to the addition of L.DNA to the in vitro transcription vessel (IVT).

As used herein, the term “quenching” refers to a process of deactivating any unreacted reagents.

Described herein are methods of producing transcribed RNA product with increased yield and reduced dsRNA impurities.

Accordingly, the present application relates to the result that the combination of increased nucleoside triphosphates (NTPs), Mg²⁺, and time led to high yields (measured in g/L) of mRNA and that such conditions resulted in much lower levels of double-stranded RNA (dsRNA) a common impurity in in vitro transcription (IVT) reactions.

### In Vitro Transcription

In vitro transcription (IVT) generally consists of a reaction that takes place in a single vessel, typically temperature controlled. IVT reactions require key components but can be enhanced by the use of one or more additives and by controlling various reaction conditions. The process generally involves the use of an engineered DNA template combined with an RNA polymerase and nucleoside triphosphates in a reaction buffer. When required by the applicable RNA polymerase of a reaction, Mg²⁺ ions are also part of the reaction mixture. Once an IVT reaction has been carried out for a sufficient amount of time, the reaction is stopped, and the crude reaction transcript can be separated and purified. The various components, additives, conditions, and other properties of IVT reactions are described in detail below. Buffers

Any suitable buffer composition can be used in an IVT process of the present disclosure. Generally, the buffer system selected for an IVT process is one that can mimic a biological environment for the enzymes used in the process and that can further facilitate the transcription reaction. Suitable buffers include, without limitation, phosphate-buffered saline (PBS) 2-(N-Morpholino)ethanesulfonic acid (MES), 2-Amino-2-hydroxymethyl-propane-1,3-diol hydrochloric acid (Tris or Tris-HCl), 4-(2-hydroxyethyl)-1-piperazineethanesulfonic acid (HEPES), 2-Bis(2-hydroxyethypamino-2-(hydroxymethyl)-1,3-propanediol (Bis-Tris), N-(2-Hydroxy-1,1-bis(hydroxymethyl)ethyl)glycine (Tricine), 3-morpholinopropane-1-sulfonic acid (MOPS), acetate, citrate, saline sodium citrate (SSC), phosphate, N-Cyclohexyl-2-aminoethanesulfonic acid (CHES), borate, [tris(hydroxymethypmethylamino]propanesulfonic acid (TAPS), 2-(bis(2-hydroxyethyl)amino)acetic acid (Bicine), 3-[N-tris(hydroxymethyl)methylamino]-2-hydroxypropanesulfonic acid (TAPSO), 2-[[1,3-dihydroxy-2-(hydroxymethyl)propan-2-yl]amino]ethanesulfonic acid (TES), or piperazine-N,N′-bis(2-ethanesulfonic acid (PIPES).

The buffer can also be in any suitable concentration. In embodiments, the buffer is in a concentration in the range of about 50 mM to about 2000 mM, about 75 mM to about 1800 mM, about 80 mM to about 1700 mM, about 85 mM to about 1600 mM, about 90 mM to about 1500 mM, about 95 mM to about 1400 mM, about 100 mM to about 1300 mM, about 125 mM to about 1200 mM, about 150 mM to about 1100 mM, about 175 mM to about 1000 mM, about 200 mM to about 900 mM, about 250 mM to about 800 mM, about 275 mM to about 700 mM, about 300 mM to about 600 mM, about 325 mM to about 550 mM, about 350 mM to about 525 mM, about 325 mM to about 575 mM, about 350 mM to about 450 mM, or about 200 mM to about 600 mM.

In embodiments, the buffer can be in a concentration of about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 375 mM, about 376 mM, about 377 mM, about 378 mM, about 379 mM, about 380 mM, about 381 mM, about 382 mM, about 383 mM, about 384 mM, about 385 mM, about 386 mM, about 387 mM, about 388 mM, about 389 mM, about 390 mM, about 391 mM, about 392 mM, about 392 mM, about 393 mM, about 394 mM, about 395 mM, about 396 mM, about 397 mM, about 398 mM, about 399 mM, about 400 mM, about 401 mM, about 402 mM, about 403 mM, about 404 mM, about 405 mM, about 406 mM, about 407 mM, about 408 mM, about 409 mM, about 410 mM, about 411 mM, about 412 mM, about 413 mM, about 414 mM, about 415 mM, about 416 mM, about 417 mM, about 418 mM, about 419 mM, about 420 mM, about 421 mM, about 422 mM about 422 mM, about 423 mM, about 424 mM, about 425 mM, about 430 mM, about 440 mM, about 450 mM, about 460 mM, about 470 mM, about 480 mM, about 490 mM, about 500 mM, about 510 mM, about 520 mM, about 530 mM, about 540 mM, about 550 mM, about 560 mM, about 570 mM, about 580 mM, about 590 mM, or about 600 mM.

### Nucleoside Triphosphates (NTPs)

RNA synthesis is catalyzed by RNA polymerase, which covalently links the free —OH group on the 3′ carbon of a growing chain of nucleotides to the α-phosphate on the 5′ carbon of the next NTP, releasing the β- and γ-phosphate groups as pyrophosphate (PPi). This results in a phosphodiester linkage between the two NTPs. The release of PPi provides the energy necessary for the reaction to occur. Typically, the NTPs in a transcription reaction are the four natural ribonucleoside triphosphates, adenosine triphosphate (ATP), uridine triphosphate (UTP), guanosine triphosphate (GTP), and cytosine triphosphate (CTP). Generally, when an in vitro transcription reaction is described in the present disclosure as being performed with NTPs without any further description, it is understood that such reaction is being carried out in the presence of ATP, UTP, GTP, and CTP. However, IVT reactions can also be carried out in the presence of one or more modified nucleoside triphosphates. For a given base-type (i.e., A, U, G, C) of an RNA transcript prepared by IVT, the transcript can be prepared in any desired molar ratio of NTPs or modified NTPs for that base-type. For example, the U-bases of an in vitro transcribed RNA can comprise 50% of natural uridine and 50% 5-methoxy uridine.

In embodiments, the IVT reactions of the present disclosure include non-natural, modified, and chemically-modified nucleotides, including any such nucleotides known in the art. Nucleotides can be artificially modified at either the base portion or the sugar portion. In nature, most polynucleotides comprise nucleotides that are “unmodified” or “natural” nucleotides, which include the purine bases adenine (A) and guanine (G), and the pyrimidine bases thymine (T), cytosine (C) and uracil (U). These bases are typically fixed to a ribose at the 1′ position. The use of RNA polynucleotides comprising chemically modified nucleotides have been shown to improve RNA expression, expression rates, half-life and/or expressed protein concentrations. RNA polynucleotides comprising chemically modified nucleotides have also been useful in optimizing protein localization thereby avoiding deleterious bio-responses such as immune responses and/or degradation pathways.

In embodiments, one or more of the nucleoside triphosphates can be chemically-modified.

Examples of modified or chemically-modified nucleotides include 5-hydroxycytidines, 5-hydroxyalkylcytidines, 5-carboxycytidines, 5-formylcytidines, 5-alkoxycytidines, 5-alkynylcytidines, 5-halocytidines, 2-thiocytidines, N4-alkylcytidines, N4-aminocytidines, N4-acetylcytidines, and N4,N4-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N4-methylcytidine, N4-aminocytidine, N4-acetylcytidine, and N4,N4-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “SMeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-taurinomethyluridine, 5-taurinomethyl-2-thiouridine, 5-(isopentenylaminomethyl)uridine, 2′-O-methylpseudouridine, 2-thio-2′-methyluridine, and 3,2′-O-dimethyluridine.

Examples of modified or chemically-modified nucleotides include N6-methyladenosine, 2-aminoadenosine, 3-methyladenosine, 8-azaadenosine, 7-deazaadenosine, 8-oxoadenosine, 8-bromoadenosine, 2-methylthio-N6-methyladenosine, N6-isopentenyladenosine, 2-methylthio-N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyl-adenosine, N6-methyl-N6-threonylcarbamoyl-adenosine, 2-methylthio-N6-threonylcarbamoyl-adenosine, N6,N6-dimethyladenosine, N6-hydroxynorvalylcarbamoyladenosine, 2-methylthio-N6-hydroxynorvalylcarbamoyl-adenosine, N6-acetyl-adenosine, 7-methyl-adenine, 2-methylthio-adenine, 2-methoxy-adenine, alpha-thio-adenosine, 2′-O-methyl-adenosine, N6,2′-O-dimethyl-adenosine, N6,N6,2′-O-trimethyl-adenosine, 1,2′-O-dimethyl-adenosine, 2′-O-ribosyladenosine, 2-amino-N6-methyl-purine, 1-thio-adenosine, 2′-F-ara-adenosine, 2′-F-adenosine, 2′-OH-ara-adenosine, and N6-(19-amino-pentaoxanonadecyl)-adenosine.

Examples of modified or chemically-modified nucleotides include Nl-alkylguanosines, N2-alkylguanosines, thienoguanosines, 7-deazaguanosines, 8-oxoguanosines, 8-bromoguanosines, O6-alkylguanosines, xanthosines, inosines, and N1-alkylinosines.

Examples of modified or chemically-modified nucleotides include N1-methylguanosine, N2-methylguanosine, thienoguanosine, 7-deazaguanosine, 8-oxoguanosine, 8-bromoguanosine, O6-methylguanosine, xanthosine, inosine, and Nl-methylinosine.

Examples of modified or chemically-modified nucleotides include pseudouridines. Examples of pseudouridines include N1-alkylpseudouridines, N1-cycloalkylpseudouridines, N1-hydroxypseudouridines, N1-hydroxyalkylpseudouridines, N1-phenylpseudouridines, N1-phenylalkylpseudouridines, N1-aminoalkylpseudouridines, N3-alkylpseudouridines, N6-alkylpseudouridines, N6-alkoxypseudouridines, N6-hydroxypseudouridines, N6-hydroxyalkylpseudouridines, N6-morpholinopseudouridines, N6-phenylpseudouridines, and N6-halopseudouridines. Examples of pseudouridines include N1-alkyl-N6-alkylpseudouridines, N1-alkyl-N6-alkoxypseudouridines, N1-alkyl-N6-hydroxypseudouridines, N1-alkyl-N6-hydroxyalkylpseudouridines, N1-alkyl-N6-morpholinopseudouridines, N1-alkyl-N6-phenylpseudouridines, and N1-alkyl-N6-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N1-methylpseudouridine (also referred to herein as “N1MPU”), N1-ethylpseudouridine, N1-propylpseudouridine, N1-cyclopropylpseudouridine, N1-phenylpseudouridine, N1-aminomethylpseudouridine, N3-methylpseudouridine, N1-hydroxypseudouridine, and N1-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-0,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-nucleotides. In an exemplary embodiment, the modified monomer is a locked nucleic acid nucleotide (LNA).

Examples of modified and chemically-modified nucleotide monomers include 2′,4′-constrained 2′-O-methoxyethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N6-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′-OH group of a nucleotide with a 2′-R, a 2′-OR, a 2′-halogen, a 2′-SR, or a 2′-amino, where R can be H, alkyl, alkenyl, or alkynyl.

Exemplary base modifications described above can be combined with additional modifications of nucleoside or nucleotide structure, including sugar modifications and linkage modifications. Certain modified or chemically-modified nucleotide monomers may be found in nature.

Preferred nucleotide modifications include N1-methylpseudouridine and 5-methoxyuridine. mRNA Caps

Only those RNA molecules that carry the Cap structure are active in Cap dependent translation; “decapitation” of mRNA results in an almost complete loss of their template activity for protein synthesis (Nature, 255:33-37, (1975); J. Biol. Chem., vol. 253:5228-5231, (1978); and Proc. Natl. Acad. Sci. USA, 72:1189-1193, (1975)).

Another element of eukaryotic mRNA is the presence of 2′-O-methyl nucleoside residues at transcript position 1 (Cap 1), and in some cases, at transcript positions 1 and 2 (Cap 2). The 2′-O-methylation of mRNA provides higher efficacy of mRNA translation in vivo (Proc. Natl. Acad. Sci. USA, 77:3952-3956 (1980)) and further improves nuclease stability of the 5′-capped mRNA. The mRNA with Cap 1 (and Cap 2) is a distinctive mark that allows cells to recognize the bona fide mRNA 5′ end, and in some instances, to discriminate against transcripts emanating from infectious genetic elements (Nucleic Acid Research 43: 482-492 (2015)).

Some examples of 5′ cap structures and methods for preparing mRNAs comprising the same are given in WO2015/051169A2, WO/2015/061491, US 2018/0273576, and U.S. Pat. Nos. 8,093,367, 8,304,529, and 10,487,105. Such methods can include cotranscriptional capping in which an RNA capping reagent that hybridizes with the linear DNA template at or near the transcription initiation site is included in the in vitro transcription reaction mixture. Another method is to have the capping done post transcriptionally through the use of enzymes that can add a the cap or methylate certain nucleotides.

In embodiments, RNA transcripts produced by the methods provided herein further comprise a 5′ cap. Any 5′ cap can be included in such RNA molecules. In further embodiments, an RNA cap may be selected from m7GpppA, m7GpppC; unmethylated cap analogs (e.g., GpppG); dimethylated cap analog (e.g., m2,7GpppG), a trimethylated cap analog (e.g., m2,2,7GpppG), dimethylated symmetrical cap analogs (e.g., m7Gpppm7G), or anti reverse cap analogs (e.g., ARCA; m7, 2′OmeGpppG, m72′dGpppG, m7,3′OmeGpppG, m7,3′dGpppG and their tetraphosphate derivatives) (see, e.g., Jemielity, J. et al., RNA 9: 1108-1122 (2003)). In additional embodiments, an RNA cap may be an ARCA cap (3′-OMe-m7G(5′)pppG). The RNA cap may be an mCAP (m7G(5′)ppp(5′)G, N7-Methyl-Guanosine-5′-Triphosphate-5′-Guanosine). The RNA cap may be resistant to hydrolysis.

### Magnesium Ion (Mg²⁺)

Generally, enzymes that have nucleotidyl transferase activity employ a general two-metal-ion mechanism to carry out an NTP condensation reaction as the NTP is adding to the elongating nucleotide strand. (Svetlov, Vladimir, and Evgeny Nudler. “Basic mechanism of transcription by RNA polymerase II.” Biochimica et Biophysica Acta vol. 1829,1 (2013): 20-8). For RNA polymerases that are used in DNA-dependent RNA transcription, the central feature of this mechanism is the employment of two magnesium cations, coordinated by at least two aspartate residues located in the active site. According to this general model the first magnesium (A) promotes deprotonation of the RNA 3′OH, facilitating 3′ O⁻ attack on the substrate NTP α-phosphate, which in turn leads to formation of a new phosphodiester bond and a leaving group, pyrophosphate (PPi). Thus, magnesium cations play an essential role in the transcription reaction.

For IVT, any suitable, water-soluble Mg²⁺ salt can be used. For example, the Mg²⁺ can be in the form of aqueous MgC₂H₃O₂ (i.e., magnesium acetate or MgOAc), MgCl₂, MgI₂, MgBr₂, and Mg(NO₃)₂. In embodiments of the IVT methods of the present disclosure, the concentration of Mg²⁺ in the transcription reaction is determined as an amount relative to the total concentration of NTPs (plus initiating cap if present). The concentration of Mg²⁺ can be at least 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM or more above the total concentration of NTPs in the reaction mixture (plus the concentration of initiating cap if present). It was found that all tested concentrations of Mg²⁺ that were at least 2 mM or more above the total concentration of NTPs in the reaction mixture (plus the concentration of initiating cap if present) is suitable to achieve increased reaction yields, and no upper limit was found to this parameter.

### DNA-Dependent RNA Polymerases

Any suitable DNA-dependent RNA polymerase can be used in the IVT methods of the present disclosure. Those skilled in the art will understand that each RNA polymerase will require a specifically matched promoter sequence on the complementary L.DNA strand to direct the RNA polymerase where to begin transcription. In order to synthesize RNA, especially large amounts of RNA, bacteriophage DNA-dependent RNA polymerase (an enzyme) is used to catalyze the transcription of RNA from a DNA template. In embodiments a Phage RNA polymerase is used. In embodiments, the RNA polymerase may be, but is not limited to T7, T3, or SP6. Bacteriophage T7 RNA polymerase is the “prototype” for other DNA-dependent RNA polymerases such as T3, SP6, and mitochondrial DNA-dependent RNA polymerases. It is also considered one of the simplest enzymes catalyzing RNA synthesis. In embodiments, the RNA polymerase is a T7 polymerase. In embodiments, the RNA polymerase is a T7 polymerase. In embodiments, the RNA polymerase is a SP6 polymerase. In embodiments, the RNA polymerase is an E. coli polymerase.

In embodiments, the amount of RNA polymerase in the transcription reaction mixture is from about 0.0125 μg/μL to about 0.15 μg/μL. In embodiments, the amount of RNA polymerase in the transcription reaction mixture is about 0.0125 μg/μL, about 0.0250 μg/μL, about 0.0375 μg/μL, about 0.0500 μg/μL, about 0.0625 μg/μL, about 0.0750 μg/μL, about 0.0875 μg/μL, about 0.1000 μg/μL, 0.1125 μg/μL, about 0.1250 μg/μL, about 0.1375 μg/μL, about 0.1500 μg/μL, about 0.1625 μg/μL, about 0.1750 μg/μL, about 0.1875 μg/μL, or about 0.2000 μg/μL. In embodiments, the range may be any interval recited between the amounts recited herein.

In embodiments, the mass ratio between RNA polymerase and linear DNA template is between 0.25 and 3.0. In embodiments, the mass ratio between RNA polymerase and linear DNA template is 0.25, 0.30, 0.35, 0.40, 0.45, 0.50, 0.55, 0.60, 0.65, 0.70, 0.75, 0.80, 0.85, 0.90, 0.95, 1.0, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.0, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, or 3.0. In embodiments, the range may be any interval recited between the mass ratios recited herein.

### DNA Linearization

As used herein, “L.DNA” refers to linearized DNA. Generally, DNA linearization is a method to produce RNA transcripts of a specified length. The DNA plasmid used as the template is linearized by a restriction enzyme downstream from the insert. In embodiments, restriction enzymes that generate 5′-overhangs may be used, and preferred, versus 3′-overhangs. Because RNA polymerases tend to “read-through” transcription, circular plasmid templates generate long heterogeneous RNA transcripts in higher quantities than linear templates. Accordingly, DNA plasmids must be completely linearized to ensure efficient synthesis of specified RNA transcript lengths. In embodiments, the application refers to “L.DNA template,” which refers to the plasmid DNA used as a template which has been linearized.

In embodiments, the amount of L.DNA template in the transcription reaction mixture is from about 0.01 mg/mL to about 0.5 mg/mL. In embodiments, the amount of L.DNA template in the transcription reaction mixture is from about 0.01 mg/mL to about 0.3 mg/mL In embodiments, the amount of L.DNA template in the transcription reaction mixture is about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.10 mg/mL, about 0.11 mg/mL, about 0.12 mg/mL, about 0.13 mg/mL, about 0.14 mg/mL, about 0.15 mg/mL, about 0.16 mg/mL, about 0.17 mg/mL, about 0.18 mg/mL, about 0.19 mg/mL, about 0.20 mg/mL, about 0.21 mg/mL, about 0.22 mg/mL, about 0.23 mg/mL, about 0.24 mg/mL, about 0.25 mg/mL, about 0.26 mg/mL, about 0.27 mg/mL, about 0.28 mg/mL, about 0.29 mg/mL, about 0.30 mg/mL, about 0.31 mg/mL, about 0.32 mg/mL, about 0.33 mg/mL, about 0.34 mg/mL, about 0.35 mg/mL, about 0.36 mg/mL, about 0.37 mg/mL, about 0.38 mg/mL, about 0.39 mg/mL, about 0.40 mg/mL, about 0.41 mg/mL, about 0.42 mg/mL, about 0.43 mg/mL, about 0.44 mg/mL, about 0.45 mg/mL, about 0.46 mg/mL, about 0.47 mg/mL, about 0.48 mg/mL, about 0.49 mg/mL, or about 0.50 mg/mL. In embodiments, the range may be any interval recited between the amounts recited herein.

### Reaction Vessels

In aspects of the present application, efficient large-scale methods are contemplated. Generally, in order to meet the demand of an efficient method, variables such as the reaction vessel or type of reaction process becomes more important. Described herein are two types of processes: (1) batch processes and (2) continuous processes each of which offers advantages and disadvantages.

A batch process refers to a process that involves a sequence of steps followed in a specific order. Batch processing involves the processing of bulk material in groups through each step of the process. Processing of subsequent batches must wait until the current is finished. While batch processing offers lower initial setup cost as an initial advantage, the overall cost of processing increases. Further, validated modeling studies confirm that the kinetics of in vitro transcription and co-transcriptional capping are equal for batch and continuous processing. In embodiments, the batch process reaction vessel can be a batch reactor.

Continuous process refers to the flow of a single unit of product between every step of the process without any break in time, substance or extent. With regard to in vitro transcription, especially as contemplated herein, continuous flow offers advantages such as space-time yield, increased speed and capacity leading to reduced lead times. In embodiments, the continuous process reaction vessel can be a continuous stirred tank reactor.

### Temperature of Reaction

In aspects of the present application, reaction temperature plays a significant role during the in vitro transcription process. Generally, higher temperatures increase the reaction rate and raise the average kinetic energy of the reactants. Further, typical in vitro transcription reactions are performed at room temperature or at 37° C. The rate of transcription decreases considerably when carried out at lower temperatures. Without being bound to any one theory, lower reaction temperatures slow the polymerase's progression, thereby preventing it from being displaced by secondary structure or a string of one specific nucleotide. In embodiments, the reaction temperature during the method of producing a transcribed RNA product is about 30° C., about 31 ° C., about 32° C., about 33° C., about 34° C., about 35° C., about 36° C., about 37° C., about 38° C., about 39° C., or about 40° C. In embodiments, the reaction temperature during the method of producing a transcribed RNA product is 30°-32° C., 31°-33° C., 32°-34° C., 33°-35° C., 34°-36° C., 35°-37° C., 36°-38° C., 37°-39° C., or 38°-40° C.

### Time of Reaction

As used herein “time of reaction” refers to incubation time of the reaction until the reaction is stopped. In aspects of the present application, reaction time plays a significant role in the quality and quantity of RNA produced in an in vitro transcription reaction. For example, the typical reaction time as contemplated herein is 4 hours, however the time may be optimized for each RNA. For transcripts shorter than 4 kb, reaction or incubation times may only be about 2 to 3 hours. For transcripts longer than 4 kb, reaction times may only be 2 hours to minimize heat exposure that can cause RNA degradation. While typical reaction times may vary in duration, extended overnight incubation is not recommended because at low nucleoside triphosphate concentrations, the T7 RNA polymerase exerts RNase activity.

In embodiments, the reaction time of in vitro transcription contemplated herein is from about 20 minutes to about 240 minutes. In embodiments, the transcription reaction time is at least 20 minutes prior to stopping the reaction. In embodiments, the reaction time is at least 40 minutes prior to stopping the reaction. In embodiments, the reaction time is from about 20 minutes to 240 minutes prior to stopping the reaction. In embodiments, the reaction time is from about 40 minutes to 240 minutes prior to stopping the reaction. In embodiments, the reaction time is from about 40 minutes to 60 minutes prior to stopping the reaction.

In embodiments, the reaction time of in vitro transcription contemplated herein is at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 115 minutes, at least 120 minutes, at least 125 minutes, at least 130 minutes, at least 135 minutes, at least 140 minutes, at least 145 minutes, at least 150 minutes, at least 155 minutes, at least 160 minutes, at least 165 minutes, at least 170 minutes, at least 175 minutes, at least 180 minutes, at least 185 minutes, at least 190 minutes, at least 195 minutes, at least 200 minutes, at least 205 minutes, at least 215 minutes, at least 220 minutes, at least 225 minutes, at least 230 minutes, at least 235 minutes, at least 240 minutes, at least 245 minutes, at least 250 minutes, at least 255 minutes, at least 260 minutes, at least 265 minutes, at least 270 minutes, at least 275 minutes, at least 280 minutes, at least 285 minutes, at least 290 minutes, at least 295 minutes, or at least 300 minutes. In embodiments, reaction times may be any interval in between 10 minutes and 300 minutes. In embodiments, reaction times may be up to 480 minutes (8 hours).

### In Vitro Transcription Termination

As used herein, “in vitro transcription termination” refers to a process of ending or stopping the reaction. Transcription termination occurs when a transcribing RNA polymerase releases the DNA template and the RNA that is being processed. Termination is required for preventing the inappropriate transcription of downstream nucleotides, and for recycling of the polymerase. There are multiple methods to stop transcription known in the art including specific sequences to stop transcription, alternate polymerases, and degrading the template. For example, if a plasmid is linearized, then the T7 polymerase has the propensity to read-through transcription or “run-off.”

The present application contemplates digesting or degrading template a method of stopping the reaction. In embodiments, the present application contemplates methods to reduce DNA contamination. In embodiments, in vitro transcription can be terminated by adding DNase I. In embodiments, DNase I is used to eliminate all genomic DNA resulting in purified RNA. In embodiments, EDTA is used in combination with DNase Ito stop in vitro transcription.

Separation of Crude and other Purification Methods

The present application contemplates highly purified mRNA. There are many techniques and methods used in the art to purify RNA products from a complex mixture, also referred to as the “crude product.” Some of these methods include, but are not limited to, precipitation, solvent extraction, ultracentrifugation, polyacrylamide gel electrophoresis, and liquid chromatography (e.g., reverse-phase ion-pairing HPLC, ion-exchange HPLC, affinity chromatography, and size-exclusion chromatography).

In an embodiment, the methods herein utilize precipitation to purify the RNA products. RNA has a negatively charged backbone which allows it to be highly soluble in water due to its polar nature. In embodiments, cations used in combination with ice-cold ethanol as a co-solvent can ionically bond to the negatively charged backbone, which reduces the solubility of the RNA such that the RNA selectively precipitates out of solution. In embodiments, the cations and their salts may be, but are not limited to, ammonium acetate and lithium chloride. The selection of the cation will depend on the size and concentration of the RNA to be precipitated.

In embodiments, the methods herein utilize solvent extraction to purify the RNA products. In embodiments, a guanidinium thiocyanate-phenol-chloroform solvent system is used to isolate and extract RNA. In embodiments, the crude mixture is incubated with an equimolar mixture of phenol and chloroform. Guanidinium thiocyanate is also a ribonuclease inhibitor. As used herein, guanidinium thiocyanate denatures the proteins and allows the proteins to be separated by the organic phase and the RNA products dissolve in the aqueous phase and thus extracted by the aqueous phase.

In embodiments, the methods herein utilize ultracentrifugation to purify the RNA products. Ultracentrifugation is especially useful for isolating large molecules such as ribosomes and ribosomal units.

In embodiments, the methods herein utilize polyacrylamide gel electrophoresis (PAGE). Polyacrylamide gel electrophoresis is a method of separating large amounts RNA products that can be applied to a variety of RNA sizes with minimal set-up and cost-effective reagents. In embodiments, polyacrylamide gel electrophoresis uses an electric field that is applied to the gel that causes the molecules to migrate based on size. A polymeric mesh is then used to separate the molecules based on size. In embodiments, the desired RNA products are isolated by excising the band from the gel. The gel is then treated to allow diffusion of the RNA products into a solution and the RNA products are extracted with an ethanolic solution.

In embodiments, the methods herein utilize liquid chromatography. In embodiments, liquid chromatography may include, but is not limited to, normal phase column chromatography (e.g., silica solid support), reverse-phase ion-pairing high-performance liquid chromatography (RP-IP-HPLC), ion-exchange high-performance liquid chromatography (IE-HPLC), ion-exchange fast-performance liquid chromatography (IE-FPLC), affinity chromatography, and size-exclusion chromatography.

In embodiments, reverse-phase ion-pairing high-performance liquid chromatography leverages the use of lipophilic cations to separate the RNA products. In embodiments, quaternary ammonium compounds ion-pair with the negatively charged sugar-phosphate backbone to afford an ion-paired complex. The ion-pair is lipophilic and then interacts with the non-polar stationary phase of the column. The desired RNA products are then eluted and separated with an organic solvent gradient. In embodiments, the organic solvent is acetonitrile.

In embodiments, ion-exchange high-performance liquid chromatography utilizes a stationary phase that contains cationic groups to create ion pairs with the negatively charged backbone of the RNA. The desired RNA product is then eluted and separated with the use of a salt gradient.

In embodiments, affinity chromatography is a separation method based on a specific binding interaction between an immobilized ligand and its binding partner. In embodiments, the immobilized ligand comprises a ligand chemically bonded or coupled to a solid support. In embodiments, the crude mixture is passed over the column, wherein those molecules having specific binding affinity to the ligand become bound. Once the impurities of the crude have been eluted, the bound molecule (analyte) is stripped from the support, resulting in its purification from the original sample. In embodiments, the RNA product may be tagged with a specific sequence to create an affinity target. In a further embodiment, the tagged RNA product may be combined with compound-activated ribozyme to cleave the RNA product of interest from the stationary phase. In embodiments, the affinity column is an oligo deoxythymine ligand coupled to a solid support.

In embodiments, size exclusion chromatography is a separation method based on differing sizes of the molecules or hydrodynamic radius of the molecules. In embodiments, the stationary phase is porous with a specific size to exclude the larger molecules and to allow the smaller molecules that fit within the pore to be retained. With regard to in vitro transcription, size exclusion chromatography is able to separate plasmid DNA from the desired RNA products. In some embodiments, the crude mixture may be first washed with phenol to extract extraneous proteins from the transcription mixture to achieve maximum purification.

### Additives

In aspects of the present application, a number of additives are used herein. In embodiments, additives may include, but are not limited to, pyrophosphatase, RNase inhibitor, solvents, calcium chloride (CaCl₂), and dithiothriotol (DTT).

### Pyrophosphatases

As used herein “pyrophosphatases” also refers to diphosphatase. Pyrophosphatases are enzymes that are acid anhydride hydrolases that hydrolyze diphosphate bonds. Pyrophosphatases are used in in vitro transcription reactions for synthesizing large-scale RNA products as it prevents pyrophosphate from precipitating with magnesium ions, which thereby increases the rate of the in vitro transcription reaction. In embodiments, the pyrophosphatase is an inorganic pyrophosphatase.

### RNase Inhibitor

The present application addresses challenges in manufacturing compositions with mRNA known to one of skill the art, including the sensitivity and instability of the molecule. There are several factors that contribute to the instability and sensitivity: (1) the presence of RNases (e.g., 5′ exonucleases, 3′ exonucleases, and endonucleases), (2) RNA is more susceptible to electrophilic additions, alkylations, and oxidations, and (3) the increased rate of hydrolysis in a solution pH exceeding 6.

As used herein “RNase inhibitor” or “RI” refers to ribonuclease inhibitor. RNase inhibitors are large molecules, approximately 49 kDa in size and rich in both cysteine and leucine compared to typical proteins. The highly-repetitive and rich leucine content allows a tight complex to form. Crystal structures of the RNase inhibitor and RNase A complex suggest that the interaction is largely electrostatic in nature for the protein-protein interactions. During in vitro transcription, RNase inhibitor protects the newly transcribed mRNA from nuclease attack.

In embodiments, the RNase inhibitor is added to the in vitro transcription mixture. In embodiments, the RNase inhibitor is added with another additive to the in vitro transcription mixture. In embodiments, the RNase inhibitor is added with inorganic pyrophosphatase. In embodiments, the RNase inhibitor is guanidium thiocyanate. In embodiments, the RNase inhibitor is guanidinium isothiocyanate. In embodiments, the RNase inhibitor can be used in an amount of about 0.10 μg/μL, about 0.11 μg/μL, about 0.12 μg/μL, about 0.13 μg/μL, about 0.14 μg/μL, about 0.15 μg/μL, about 0.16 μg/μL, about 0.17 μg/μL, about 0.18 μg/μL, about 0.19 μg/μL, about 0.20 μg/μL, about 0.21 μg/μL, about 0.22 μg/μL, about 0.23 μg/μL, about 0.24 μg/μL, about 0.25 μg/μL, about 0.26 μg/μL, about 0.27 μg/μL, about 0.28 μg/μL, about 0.29 μg/μL, or about 0.30 μg/SEL.

### Solvents

In aspects of the present application, additives such as an organic solvent are contemplated.

In embodiments, the solvent is selected from a polar protic solvent. In embodiments, the polar protic solvent is selected from the group consisting of water, methanol, ethanol, and isopropanol. In embodiments, the solvent is selected from a polar aprotic solvent. In embodiments, the polar aprotic solvent is selected from acetonitrile. In embodiments, the solvent is selected from methanol (MeOH), ethanol (EtOH) , isopropanol (i-PrOH), acetonitrile (CH₃CN or MeCN), and combinations thereof

In embodiments, the concentration of ethanol used as a solvent in the reaction mixture of RNA transcription is from about 1% v/v to about 10% v/v. In embodiments, the concentration of ethanol used as a solvent in the reaction mixture of RNA transcription is about 1% v/v, about 2% v/v, about 3% v/v, about 4% v/v, about 5% v/v, about 6% v/v, about 7% v/v, about 8% v/v, about 9% v/v, or about 10% v/v.

In embodiments, the concentration of methanol used as a solvent in the reaction mixture of RNA transcription is from about 1% v/v to about 10% v/v. In embodiments, the concentration of methanol used as a solvent in the reaction mixture of RNA transcription is about 1% v/v, about 2% v/v, about 3% v/v, about 4% v/v, about 5% v/v, about 6% v/v, about 7% v/v, about 8% v/v, about 9% v/v, or about 10% v/v.

In embodiments, the concentration of isopropanol used as a solvent in the reaction mixture of RNA transcription is from about 1% v/v to about 10% v/v. In embodiments, the concentration of isopropanol used as a solvent in the reaction mixture of RNA transcription is about 1% v/v, about 2% v/v, about 3% v/v, about 4% v/v, about 5% v/v, about 6% v/v, about 7% v/v, about 8% v/v, about 9% v/v, or about 10% v/v.

In embodiments, the concentration of acetonitrile used as a solvent in the reaction mixture of RNA transcription is from about 1% v/v to about 8% v/v. In embodiments, the concentration of acetonitrile used as a solvent in the reaction mixture of RNA transcription is about 1% v/v, about 2% v/v, about 3% v/v, about 4% v/v, about 5% v/v, about 6% v/v, about 7% v/v, or about 8% v/v.

### Role of CaCl₂

As used herein “DNase I” refers to an deoxyribonuclease I, which is an endonuclease that non-specifically cleaves single- and double-stranded DNA. It hydrolyzes phosphodiester bonds producing mono- and oligodeoxyribonucleotides with 5′-phosphate and 3′-OH groups. Calcium chloride, which provides Ca^{2 +}, is an additive that has been found to be required for DNase I activity with Mg²⁺. Without being bound to any one theory, it is suggested that Ca²⁺ ions play an important role in the structural integrity of DNase I, whereas other metal cations such as Mg²⁺ and Mn²⁺ bind to the DNA substrate itself (Pan, C. Q. and Lazarus, R. A. Protein Science 1999, 8, 1780-1788). In crystal structures of bovine DNase I, “there are two distinct Ca²⁺ binding sites that stabilize two surface loops as well an additional metal ion binding site at the active site.” (Pan and Lazarus, 1999 citing Oefner C. and Suck D. J Mol. Biol. 1986, 192, 605-632.) Accordingly, the presence of calcium chloride was found to play an integral part in in vitro transcription as contemplated herein.

### DTT

As used herein, “DTT” refers to dithiothreitol or Cleland's reagent, an additive contemplated herein for in vitro transcription. DTT is a reducing agent used to reduce disulfide bonds of proteins to prevent intramolecular and intermolecular disulfide bonds forming between the cysteine residues of proteins. In embodiments, DTT is used to prevent dimerization. In embodiments, DTT is used as an additive to improve separation of proteins during electrophoresis by denaturing proteins.

### Guinidine Hydrochloride

As used herein, “GnCl” refers to guanidine hydrochloride, a denaturant used in in vitro transcription to improve the processive transcriptional activity of the T7 RNA polymerase enzyme.

### EMBODIMENTS

In an aspect, the present application provides a method of producing a transcribed RNA product comprising:
a. reacting a transcription reaction mixture comprising a buffer solution comprising [Mg²⁺], linear DNA (L.DNA) template, ribonucleoside tri-phosphate (rNTPs), optionally an RNA capping reagent, and RNA polymerase; wherein a molar concentration of Mg²⁺ is 2 to 15 mM above the total molar concentration of all rNTPs plus the optional RNA capping reagent; and a RNA polymerase/linear DNA template mass ratio is between 0.25 and 3;
b. stopping the transcription reaction by digesting the L.DNA template with deoxyribonuclease (DNase) or quenching the enzyme with ethylenediaminetratracetic acid (EDTA);
wherein a yield of about 1 g/L to about 25 g/L of single stranded transcribed RNA divided by the volume of liquid of the transcription reaction prior to the addition of DNase in step (b) is produced.

In an embodiment, the transcription reaction in the step (b) is stopped by digesting the L.DNA template with DNase.

In an embodiment, the reaction in the step (b) is stopped by quenching the enzyme with EDTA.

In an embodiment, the L.DNA template is in a solution comprising 50 mM to 1200 mM NaCl to produce a salt-spiked L.DNA template prior to introduction to the transcription reaction mixture.

In an embodiment, the salt-spiked L.DNA template is in a solution comprising 200 mM to 1000 mM NaCl, prior to introduction to the transcription reaction mixture.

In an embodiment, the transcription reaction mixture of step (a) further comprises one or more of the group consisting of RNase inhibitor and inorganic pyrophosphatase.

In an embodiment, the pH of the reaction mixture is a range from 6.5 to 8.0.

In an embodiment, the temperature during step a) and b) is a range from 30° C. to 40° C.

In an embodiment, the L.DNA template is from about 0.01 mg/mL to about 0.3 mg/mL mM in the transcription reaction mixture

In an embodiment, the RNA polymerase is T7 polymerase.

In an embodiment, a T7 polymerase KU activity per mg of L.DNA is greater than or equal to 125 KU T7 polymerase activity.

In an embodiment, the transcription reaction mixture of step (a) is allowed to react for at least 20 minutes prior to step (b) stopping the transcription reaction.

In an embodiment, the transcription reaction mixture of step (a) is allowed to react for at least 40 minutes prior to stopping the reaction by adding DNase I in step (b).

In an embodiment, the transcription reaction mixture of step (a) is allowed to react for 40 to 240 minutes prior to stopping the reaction in step (b).

In an embodiment, the transcription reaction mixture of step (a) is allowed to react for 40 to 60 minutes prior to stopping the reaction in step (b).

In an embodiment, the transcription mixture of step (a) further comprises a transcription initiating RNA capping reagent.

In an embodiment, the RNA cap is an anti reverse cap analog (ARCA cap).

In an embodiment, the method further comprises a post-transcriptional capping step.

In an embodiment, the buffer solution of step (a) further comprises one or more buffers selected from tris(hydroxymethyl)aminomethane (TRIS) and (4-(2-hydroxyethyl)-1-piperazineethanesulfonic acid (HEPES.)

In an embodiment, the molar concentration of Mg²⁺ is 5 to 15 mM above the total molar concentration of all rNTPS plus the molar concentration of any optional RNA capping reagent.

In an embodiment, the molar concentration of Mg²⁺ is about 7 to 10 mM above the total molar concentration of all rNTPS plus the molar concentration of any optional RNA capping reagent.

In an embodiment, the RNA polymerase is 0.0125 to 0.15 μg/μL T7 polymerase.

In an embodiment, the reaction produces 3 to 20 g/L of single stranded transcribed RNA.

In an embodiment, the reaction produces 5 to 16 g/L of single stranded transcribed RNA.

In an embodiment, the amount of single stranded transcribed RNA is measured after purification of the transcription mixture after step (b) via a silica column.

In an embodiment, the amount of single stranded transcribed RNA is measured after purification of the transcription mixture after step (b) via affinity column chromatography. In an embodiment, the affinity column chromatography is an oligo deoxthymine ligand coupled to a solid support.

In an embodiment, purification of the transcription mixture after step (b) via a silica column is performed before purification via affinity column chromatography.

In an embodiment, the RNA transcription mixture of step (a) further comprises one or more solvents selected from the group consisting of EtOH in a concentration of 1 to 10% v/v, PrOH in a concentration of 1 to 10% v/v, MeOH in a concentration of 1 to 10% v/v, and i-acetonitrile of a concentration of 1 to 8% v/v, with the proviso that the total concentration of EtOH, i-PrOH, MeOH and acetonitrile does not exceed 10% v/v of the RNA transcription mixture of step (a).

In an embodiment, the RNA transcription mixture of step (a) comprises 1 to 10% (v/v) EtOH.

In an embodiment, the RNA transcription mixture comprises 5% (v/v) EtOH.

In an embodiment, the RNA transcription mixture of step (a) comprises 1 to 10% (v/v) acetonitrile.

In an embodiment, the RNA transcription mixture comprises 5% (v/v) acetonitrile

In an embodiment, the RNA transcription mixture of step (a) comprises 1 to 10% (v/v) MeOH.

In an embodiment, the RNA transcription mixture comprises 7% (v/v) MeOH.

In an embodiment, the RNA transcription mixture of step (a) comprises 1 to 10% (v/v) iPrOH.

In an embodiment, the RNA transcription mixture comprises 5% (v/v) iPrOH.

In an embodiment, the L.DNA template comprises an open reading frame that encodes a vaccine antigen, enzyme, antibody, receptor, tRNA, and/ or a protein.

In an embodiment, the total concentration of rNTPs is at least 8 mM

In an embodiment, a yield of greater than about 5 g/ L of RNA transcript results in a reduced amount of dsRNA as compared to an otherwise identical transcription reaction wherein the yield is less than about 5 g/L.

In an aspect, the present application provides a method of transcribing RNA product comprising:
a. loading a reactor vessel with a buffer solution comprising Mg²⁺ and nuclease free water;
b. adding a solution comprising L.DNA template spiked with 200-1000 mM NaCl;
c. adding rNTPs (define as wild-type and analogs) and optionally RNA cap (define in specification);
d. adding RNA polymerase and mixing the resulting transcription solution wherein the RNA polymerase/DNA template mass ratio is between 0.25 and 3; and,
e. adding DNase to the transcription solution;
wherein the molar concentration of Mg²⁺ is 2 to 15 mM above the total molar concentration of all rNTPs plus the optional RNA cap after steps (a)-(b) are completed, and
wherein 1 to 25 g of single stranded transcribed RNA per liter of solution of the transcription reaction prior to the addition of DNase in step (e) is produced.

In an embodiment, a yield of greater than about 5 g/L of RNA transcript results in a reduced amount of dsRNA as compared to an otherwise identical transcription reaction wherein the yield is less than about 5 g/L.

In an aspect, the present application provides a method of producing a transcribed RNA product with reduced dsRNA comprising:
a. reacting a transcription reaction mixture comprising a buffer solution comprising Mg²⁺, L.DNA template previously spiked with 50 to 1200 mM NaCl, rNTPs, optionally RNA cap, RNA polymerase wherein the RNA polymerase/L. DNA template mass ratio is between 0.25 and 3, and one or more solvents selected from the group consisting of EtOH in a concentration of 1 to 10% v/v, i-PrOH in a concentration of 1 to 10% v/v, MeOH in a concentration of 1 to 10% v/v, and acetonitrile of a concentration of 1 to 8% v/v, with the proviso that the total concentration of EtOH, i-PrOH, MeOH and acetonitrile does not exceed 10+/−1% v/v of the RNA transcription mixture; and,
b. adding DNase to the transcription reaction mixture;
wherein the molar concentration of Mg²⁺ in the transcription reaction mixture is 2 to 15 mM above the total molar concentration of all rNTPs plus the optional RNA cap.

In an embodiment, the amount of dsRNA is reduced compared to an otherwise identical transcription reaction wherein the transcription reaction mixture excludes added EtOH, i-PrOH, MeOH, and/or acetonitrile.

### EXAMPLES

The following examples are offered for purposes of illustration, and are not intended to limit the scope of the claims provided herein. All literature citations in these examples and throughout this specification are incorporated herein by references for all legal purposes to be served thereby.

### Example 1

In Vitro Transcription (IVT) for Synthesis of mRNA

An IVT reaction vessel was loaded with nuclease-free water and 10× IVT buffer solution containing 400 mM TRIS, pH 7.5, and varying amounts of MgOAc as listed in Table 2 below. Dithiothreitol, salt spiked linearized plasmid DNA (L. DNA) template, rNTP mix (these rNTPs include ATP, CTP, GTP, UTP, and/or modified NTPs), and optional capping reagent (for those reactions listed that used it), were added to the reaction vessel, and allowed to equilibrate to 37±2° C. Then T7 RNA polymerase, RNase inhibitor (RI), and pyrophosphatase (IPPase) were added, and the resulting mixture was incubated for 20 to 240 minutes at about 37° C. The amounts for the reagents used in this reaction other than those recited above are listed in Table 1. The transcription reaction was followed by DNase reaction by adding DNase I enzyme with the buffer containing TRIS, MgCl₂ and CaCl₂ at about 37° C. The reaction was stirred for about 15 -20 minutes. The DNase reaction was quenched by adding EDTA (ethylenediaminetetraacetic acid.) The resulting mixture was purified using silica column purification. Yield was calculated via UV spectrophotometry at A260. The presence of dsRNA impurities was determined via the Dot blot method described in Example 2.

When additives that decreased dsRNA, such as ethanol and acetonitrile, were used, they were added after the nuclease-free water and 10× IVT buffer were heated to 37° C., right before adding the L. DNA.

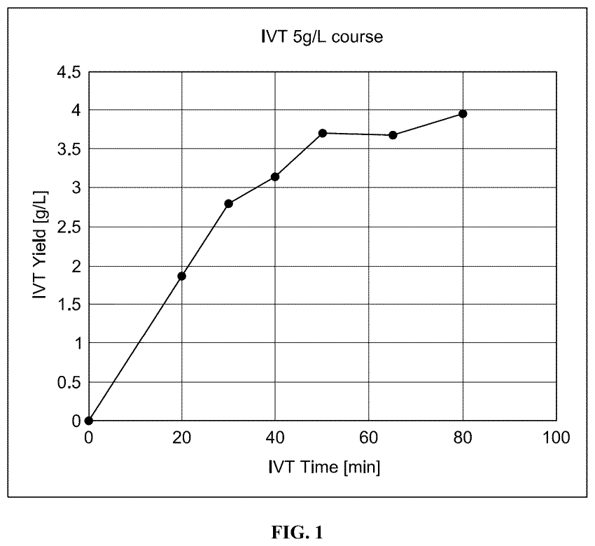

Table 1 shows the amounts of each raw material, additive and enzyme added to perform a 200 μL IVT reaction that afforded 5 g/L, or 1 mg of mRNA in total. This reaction is referred to below as the 5 g/L reaction. The results of the reaction of Table 1 are shown in FIG. 1.

TABLE 1
IVT reaction detail for the experiments described in Example 1.

| | Final Concentration | |
|---|---|---|
| Buffer | 40 mM Tris, 30 mM MgAc | |
| L.DNA | 0.075 | mg/ml |
| DTT* | 10 | mM |
| ATP | 5 | mM |
| CTP | 5 | mM |
| GTP | 5 | mM |
| UTP | 5 | mM |
| Capping reagent | 1.5 | mM |
| Rnase Inhibitor | 0.25 | μg/μL |
| IPP# | 0.002 | U/μL |
| T7 Polymerase | 0.025 | mg/mL |

| | |
|---|---|
| Time, Temp | 50 min, 37° C. |

*DTT = dithiothreotol
#IPP = pyrophosphatase

Notably, T7 RNA polymerase was procured from Roche, where each lot would have a given amount of proteins (1.0 mg/mL) but varying amount of specific activity (Specification on Volume Activity is ≥1000 KU/mL; equivalent to Specific Activity of ≥1000 KU/mgP).

Three reactions were conducted varying parameters with the goal of achieving a desired target yield: 5 g/L, 10 g/L, and 15 g/L. Table 2 describes the key differences between the conventional (5 g/L IVT reaction condition) and the other two high-yield reaction conditions (yields as high as 10 g/L and 15 g/L were achieved under these conditions). Unless otherwise specified in Table 2, the reagent amounts of Table 1 above remained unchanged across these different methods.

TABLE 2
IVT reaction differences

| | Final Concentrations | | |
|---|---|---|---|
| IVT Target Yield | 5 g/L | 10 g/L | 15 g/L |
| MgOAc (mM) | 30 | 50 | 65 |
| ATP (mM) | 5 | 10 | 15 |
| CTP (mM) | 5 | 10 | 15 |
| GTP (mM) | 5 | 10 | 15 |
| UTP (mM) | 5 | 10 | 15 |

### Post- and Co-Transcriptional Capping

A. Enzymatic Capping of IVT mRNA

Messenger RNA transcripts of the present disclosure can be capped by any suitable means including by post-transcriptional enzymatic capping or by cotranscriptional capping. For enzymatic capping, a scaled-up version (50-times larger) of New England BioLabs® (NEB's) one-step capping and 2′-O-methylation reaction was used, that was suitable for treating up to 1 mg of IVT transcripts. A 10 μg RNA in a 20 μL reaction was recommended, based on the assumption that transcript length would be as short as 100 nt. However, a higher substrate-to-reaction volume was acceptable for mRNA transcripts, which were generally longer (about 1,000-15,000 nt) in length. Before initiating the capping reaction, the RNA was denatured at 65° C. for 5 minutes and then immediately put on ice to relieve any secondary conformations. For the total 1 mL capping reaction, 1 mg denatured RNA in 700 μL of nuclease-free water was used along with 100 μL (10×) capping buffer, 50 μL (10 mM) GTP, 50 μL (4 mM) SAM, 50 μL of (10 U/μL) Vaccinia capping enzyme and 50 μL of mRNA cap 2′-O-methyltransferase at (50 U/μL) were combined and incubated at 37° C. for 1 hour. The resulting capped mRNA was eluted using RNase free water, re-purified on an RNeasy column, and then quantified by nanodrop. The mRNA was also visualized on the gel by running 500 ng of the purified product per lane in a denaturing gel after denaturation and immediately put on ice to remove secondary structures.

### B. Co-Transcriptional Capping

For co-transcriptional capping with the Anti-Reverse Cap Analog (ARCA) cap, a scaled-up version of New England Biolab's® (“NEB's”) protocol was used. A 10 μg RNA was mixed with 100 μL of 2× ARCA/NTP mix, 20 μL of T7 RNA polymerase mix, and 1.7 μL of IPPase and RI. Nuclease-free water was added to a total volume of 200 μL. The reaction mixture was well mixed and incubated for 30 min at 37° C. After performing DNase I reaction, the mRNA was purified and quantified.

### Silica Purification

1 mg of RNA sample was diluted with a mixture of nuclease-free water, β-mercaptoethanol, guanidium thiocyanate, and ethanol. The mixture was loaded on a Nucleospin® Blood XL column and centrifuged at 4000×g for 2 minutes at ambient temperature. The column was washed two times in equal volumes with a solution that contained ethanol and guanidinium thiocyanate, and the purified mRNA was eluted with WFI. Silica purification was used to assess the concentration of the IVT reactions ranging from 200 μL to 20 Liters. The in vitro transcribed RNA was analyzed for dsRNA impurities using Dot Blots as described in Example 2.

### Oligo dT Purification

To measure yield, the IVT pool was further purified using a BIA Separations oligo-dT column. The oligo-dT column contained a dTis oligomer as a ligand, which hybridizes with the mRNA polyA tail. Binding and washing conditions used high salt sodium phosphate/NaCl buffers and elution using WFI. Impurities such as enzymes, free NTPs, digested DNA, and abortive mRNAs were removed, and purified mRNA was collected in the WFI elution fraction. Methods used in oligo dT purification are further described in U.S. 2019/0203199, the entire contents of which are incorporated herein by reference.

### (i). Increasing NTP Concentration Alone Does Not Increase Yield

Using the above process of Example 1 on a 200 μl -scale (Table 1), with a 11000 nt replicon as a target, with 0.075 mg/mL of L. DNA spiked with 200 mM NaCl, (5 mM of each NTP, 1.5 mM RNA cap, 30 mM magnesium acetate (7 mM excess), 0.025 μg/μL T7 polymerase, 40 mM Tris, pH 7.6, 10 mM DTT, 0.25 U/ μL RNase inhibitor, and 0.002 U/μL, PPase, the target yield of 5 g/L (±1 g/L yield variation) was obtained after 50 minutes as shown in FIG. 1, which displays the IVT yield over the time of reaction for a reaction with a targeted 5 g/L. Similar results were obtained with L. DNAs of various sizes (Table 3).

Table 3 lists the different mRNA types that were tested using the old (5 g/L) and new (10 or 15 g/L) conditions of the present disclosure. Tested mRNAs covered a range of sizes, chemistry, self-replicating vs. non-replicating, capped vs uncapped, with and without poly(A) tail, in addition to vaccine and therapeutic targets.

TABLE 3
Summary of Example 1

| | 696 nt-1429 nt |
|---|---|
| size | 696 nt (lunar OVA, 14291 nt (Lunar CV mRNA) |
| chemistry | Wild type, N1-methyl pseudo-uridine |
| type | Standard mRNA and self-replicating replicons, tRNA, circular |
| | RNA |
| Capped | +/− cap, tested with different cap analogs |
| poly A | Works for constructs with +/− poly A tails |
| Target | Vaccine immunogens (Covid Spike protein, flu HA), receptors |
| | (CFTR, LDLR), enzymes (OTC, PAH), Talens, tRNA |

Analysis of the reaction mixture for the targeted 5 g/L reaction conditions revealed that NTPs were depleted from the IVT reaction mixture, which suggested that yield could be increased by increasing NTPs. Using the above process of Example 1 on the 200 μL-scale reaction, with a 11000 nt replicon as a target, with 0.075 mg/mL of L. DNA spiked with 200 mM NaCl, 10 mM of each NTP, 1.5 mM RNA cap, 30 mM magnesium acetate (11.5 mM deficient), 0.025 μg/μL T7 polymerase, 40 mM Tris pH 7.6, 10 mM DTT, 0.25 μg/μL RNAse inhibitor (RI), and 0.002 U/μL pyrophosphatase (PPase), only 0.6 g/L was obtained after 60 minutes, whereas a yield of 10 g/L was targeted. Similar results were obtained with L. DNA's of various sizes.

This proved that merely increasing the NTP concentration alone does not improve the IVT reaction yield.

### (ii). Increasing Mg²⁺ Concentration and NTPs Concentration Does Not Achieve the IVT Targeted Reaction Yield at the 60 Minute Time Point

Without being bound to any theory, it was hypothesized that the low yields obtained after increasing NTPs may have been due to depletion of Mg²⁺ from the solution, as each NTP molecule associates with a Mg²⁺ atom. Therefore, together with increasing the NTPs to 10 mM each, the Mg²⁺ concentration was increased from 30 mM to 50 mM, which was in excess of 8.5 mM. Using the above process of Example 1 on the 200 μL-scale reaction, with a 11000 nt replicon as a target, with 0.075 mg/mL of L. DNA spiked with 200 mM NaCl, 10 mM of each NTP, 1.5 mM RNA cap, 50 mM magnesium acetate (8.5 mM excess), 0.025 μg/μL T7 polymerase, 40 mM Tris pH 7.6, 10 mM DTT, 0.25 μg/μL RI, and 0.002 U/μL PPase surprisingly only yielded 7.1 g/L after 60 minutes. Similar results were obtained with other L. DNAs of various sizes.

Thus, adjusting the reaction conditions to further increase Mg²⁺ concentration did not achieve the targeted IVT reaction yields.

(iii). Longer Reaction Time with Increased NTP and Mg²⁺ concentration Produces Increased Yield

To investigate whether the expected IVT yields could be achieved by increasing both NTPs and Mg²⁺ concentration, the time course of the reaction was studied.

Using the above process of Example 1 on the 200 μL scale, with a 11000 nt replicon as the encoded transcript, with 0.075 mg/mL of L. DNA spiked with 200 mM NaCl, 10 mM of each NTP, 1.5 mM RNA cap, 50 mM magnesium acetate (8.5 mM excess), 0.025 μg/μL T7 polymerase, 40 mM Tris pH 7.6, 10 mM DTT, 0.25 μg/μL RI, and 0.002 U/μL PPase, a yield of 9.5 g/L was obtained after extending the IVT time from 60 to 90 minutes. Similar results were obtained with the L. DNAs of various sizes.

Likewise, using the above process of Example 1 on a 200 μL-scale, with a 11000 nt replicon as the encoded transcript, with 0.075 mg/mL of L. DNA spiked with 200 mM NaCl, 15 mM of each NTP, 1.5 mM RNA cap, 65 mM magnesium acetate (3.5 mM excess), 0.025 μg/μL T7 polymerase, 40 mM Tris pH 7.6, 10 mM DTT, 0.25 μg/μL RI, and 0.002 U/μL PPase, a yield of 15 g/L was obtained after 240 minutes. Similar results were obtained with the L. DNAs of various sizes.

10 g/L larger scale: the above process of Example 1 was applied on a 50 mL-scale IVT volume, with a 11000 nt replicon as the encoded transcript, with 0.075 mg/mL of L. DNA spiked with 200 mM NaCl, 10 mM of each NTP, 1.5 mM RNA cap, 50 mM magnesium acetate (8.5 mM excess), 0.025 μg/μL T7 polymerase, 40 mM Tris, pH 7.6, 10 mM DTT, 0.25 μg/μL RI, and 0.002 U/μL PPase, and obtained a yield of 10.5 g/L at 90 minutes.

15 g/L larger scale: likewise, using the above process of Example 1 on a 50 mL-scale IVT, with a 11000 nt replicon as the encoded transcript, with 0.075 mg/mL of L. DNA spiked with 200 mM NaCl, 15 mM of each NTP, 1.5 mM RNA cap, 65 mM magnesium acetate (3.5 mM excess), 0.025 μg/μL T7 polymerase, 40 mM Tris pH 7.6, 10 mM DTT, 0.25 μg/μL RI, and 0.002 U/μL PPase, a yield of 15 g/L was obtained after 240 minutes. Similar results were obtained with the L. DNAs of various sizes.

Table 4 shows the summary of the experiments described above and the applicable outcome (yield and dsRNA levels).

TABLE 4
Summary of Experiments Testing Different Polynucleotide Sizes and dsRNA Levels

| L.DNA | | L.DNA | | | | | | | |
|---|---|---|---|---|---|---|---|---|---|
| template | L.DNA | NaCl | Mg²⁺ | NTP | Cap | T7 | Reaction | Yield | |
| size | Conc. | conc. | conc. | amounts | amount | pol. | Time | (g/L) | dsRNA |
| 11000 | 0.075 | 200 | 30 | 5 mM | 1.5 | 0.025 | 50 | 5 | 100% |
| nt | mg/ml | mM | mM | each | mM | μg/μL | min | g/L | ref1* |
| 11000 | 0.075 | 200 | 30 | 10 mM | 1.5 | 0.025 | 60 | 0.6 | ND |
| nt | mg/ml | mM | mM | each | mM | μg/μL | min | g/L | |
| 11000 | 0.075 | 200 | 50 | 10 mM | 1.5 | 0.025 | 60 | 7.1 | ND |
| nt | mg/ml | mM | mM | each | mM | μg/μL | min | g/L | |
| 11000 | 0.075 | 200 | 50 | 10 mM | 1.5 | 0.025 | 90 | 9.5 | 21.9% |
| nt | mg/ml | mM | mM | each | mM | μg/μL | min | g/L | of ref1 |
| 11000 | 0.075 | 200 | 65 | 15 mM | 1.5 | 0.025 | 240 | 15 | 11.1% |
| nt | mg/ml | mM | mM | each | mM | μg/μL | min | g/L | of ref1 |
| 14291 | 0.075 | 400 | 30 | 5 mM | 1.5 | 0.025 | 50 | 5.1 | 100% |
| nt | mg/ml | mM | mM | each | mM | μg/μL | min | g/L | ref2** |
| 14291 | 0.075 | 400 | 50 | 10 mM | 1.5 | 0.075 | 180 | 13 | 18-30% |
| nt | mg/ml | mM | mM | each | mM | μg/μL | min | g/L | of ref2 |
| 696 | 0.075 | 400 | 30 | 5 mM | 1.5 | 0.025 | 50 | 5.9 | 100% |
| nt | mg/ml | mM | mM | each | mM | μg/μL | min | g/L | ref3*** |
| 696 | 0.075 | 400 | 50 | 10 mM | 1.5 | 0.075 | 180 | 9.2 | 23% of |
| nt | mg/ml | mM | mM | each | mM | μg/μL | min | g/L | ref3 |

*= level of dsRNA of the 11000 nt construct at the 5 g/L condition
**= level of dsRNA of the 14291 nt construct at the 5 g/L condition
***= level of dsRNA of the 696 nt construct at the 5 g/L condition

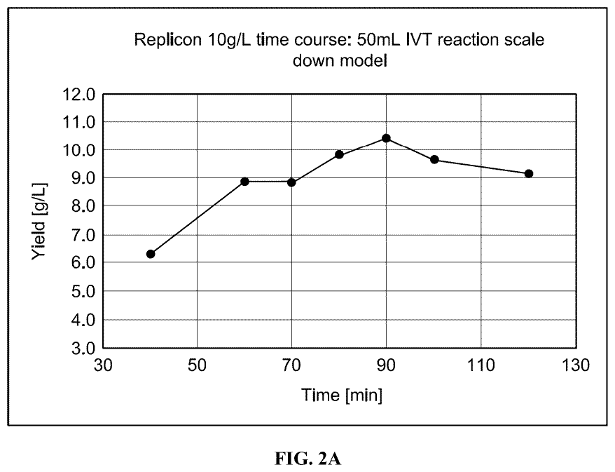

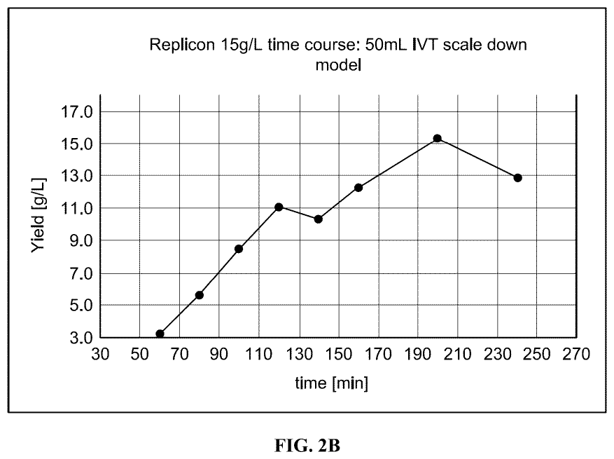

FIG. 2a shows the yield over time for reactions conducted at the 10 g/L target and FIG. 2b shows the yield over time for reactions conducted at the 15 g/L target. It can be seen that for both conditions, the yield continues to increase above reaction times of 60 min.

Conclusions: The IVT yield was dictated by the amount of NTPs (building blocks), Mg²⁺ concentration and time. An increase in NTPs alone was not effective to achieve high yields. Increased NTPs must be accompanied by an increase in Mg²⁺ concentration and time. Mg²⁺ must stay above the total molarities of all NTPs (including cap) and time has to increase because higher yield reactions have slower kinetics. Increasing the IVT yield must have the increase of NTPs/Mg²⁺/time trio together, and anything missing of this trio will not result in achieving the targeted yield.

### Example 2 dsRNA Reduction

dsRNA Quantification by Dot Blots

mRNA samples (100 ng) were dotted on each mRNA Biodyne® pre-cut modified nylon membrane (Thermo Scientific, Catalog #77016) (0.45 m, 8×12 cm). The membrane was blocked by incubating 5% non-fat dried milk in TBS-T buffer (50 mM Tris HCl, 150 mM NaCl (pH 7.4) and 0.05% Tween-20®) for 1 hour, and then was incubated with primary antibody anti-dsRNA mAB J2 (English and Scientific Consulting K ft., Hungary, J2 monoclonal antibody (mAb), mouse, IgG2a, Batch # J2-1507, 1.0 mg/mL). After 1 hr incubation time, the membrane was washed using TBS-T buffer, each for 7 mins (4×7 min). Then the membrane was incubated with secondary antibody (Life Technologies, Goat anti-mouse IgG, (H+L), HRP Conjugate, Catalog #16066) for 1 hour at room temperature, followed by washing 6 times with TBS-T (6×5 min), then once with TBS (5 min). The resulting membrane was incubated with ECL reagent (SUPERSIGNAL WEST PICO AND FEMTO MIX, Thermo Scientific, Catalog #34080 and 34095) for 3-4 min and exposed under white light inside Chemidoc-It² Imaging System. Intensities of sample dots per mRNA load were compared to dot intensities generated from a series of Poly(I:C) standards or reference samples. Poly(I:C) forms double stranded RNA, where one strand is inosinic acid and the other is cytidylic acid.

dsRNA quantification by ELISA

Samples of mRNA produced by IVT were diluted in water, prepared in buffers, and then transferred to a Nunc MaxiSorp Flat Bottom Plate (Invitrogen 44-2404-21) plate pre-coated with dsRNA specific antibodies (Primary antibody GenScript Al monoclonal antibody (mAB), mouse, IgG2a, Full Length Antibody, Kappa Light Chain (GenScript Lot# US3177EG180-1/P7EH011)) and pre-blocked with a TBST, 1% BSA buffer to avoid non-specific binding and to decrease background noise. The plate was then washed with TBST, and dsRNA was detected using an HRP-conjugated primary antibody. The primary antibody binds specifically to dsRNA. The plate was then washed 3× with TBST, and was then treated with TMB substrate which binds to HRP to induce a color change, after 10 minutes at ambient temperature, the reaction was quenched using sulfuric acid. All absorbance values of signal were measured at 450 nm. The absorbance values of samples per mRNA load were compared to absorbance values from a series of Poly(I:C) standards or a series of dsRNA reference standards. Poly I:C forms double-stranded RNA where one strand is inosinic acid and the other cytidylic acid.

(iv). Increasing IVT Yield Also Reduces dsRNA

Increasing the NTPs, Mg²⁺ concentration, and time to achieve higher yields also reduces dsRNA impurities by 5 to 10-fold.

The general process outlined in Example 1 was applied for IVT reactions targeting 10 g/L and 15 g/L yields on 200 μL (small scale) and 50 mL (large scale) using the parameters outlined in Table 5 below. Silica gel purification and then Dot Blot were performed to assess the amount of dsRNA, a key impurity in the IVT reactions.

TABLE 5
IVT parameters used for the 50 mL IVT and 200 μl scale down model reaction

| L.DNA | | L.DNA | | | | | | | |
|---|---|---|---|---|---|---|---|---|---|
| template | | NaCl | Mg²⁺ | NTP | Cap | T7 | Reaction | Yield | |
| with size | L.DNA | conc. | conc. | amounts | amount | pol. | Time | (g/L) | dsRNA |
| 11000 | 0.075 | 200 | 50 | 10 mM | 1.5 | 0.0375 | 90 | 9.5 | 9% |
| nt (50 ml | mg/mL | mM | mM | each | mM | μg/μL | min | g/L | of ref* |
| scale, | | | | | | | | | |
| target 10 | | | | | | | | | |
| g/L) | | | | | | | | | |
| 11000 | 0.075 | 200 | 50 | 10 mM | 1.5 | 0.0375 | 90 | 9.5 | 21.9% |
| nt (200 μl | mg/mL | mM | mM | each | mM | μg/μL | min | g/L | of ref |
| scale, | | | | | | | | | |
| target 10 | | | | | | | | | |
| g/L) | | | | | | | | | |
| 11000 | 0.075 | 200 | 65 | 15 mM | 1.5 | 0.0375 | 240 | 15 | 6.7% |
| nt (50 ml | mg/mL | mM | mM | each | mM | μg/μL | min | g/L | of ref |
| scale, | | | | | | | | | |
| target 15 | | | | | | | | | |
| g/L) | | | | | | | | | |
| 11000 | 0.075 | 200 | 65 | 15 mM | 1.5 | 0.0375 | 240 | 15 | 11.1% |
| nt (200 μl | mg/mL | mM | mM | each | mM | μg/μL | min | g/L | of ref |
| scale, | | | | | | | | | |
| target 15 | | | | | | | | | |
| g/L) | | | | | | | | | |

*= level of dsRNA of the 11000 nt construct at the 5 g/L condition

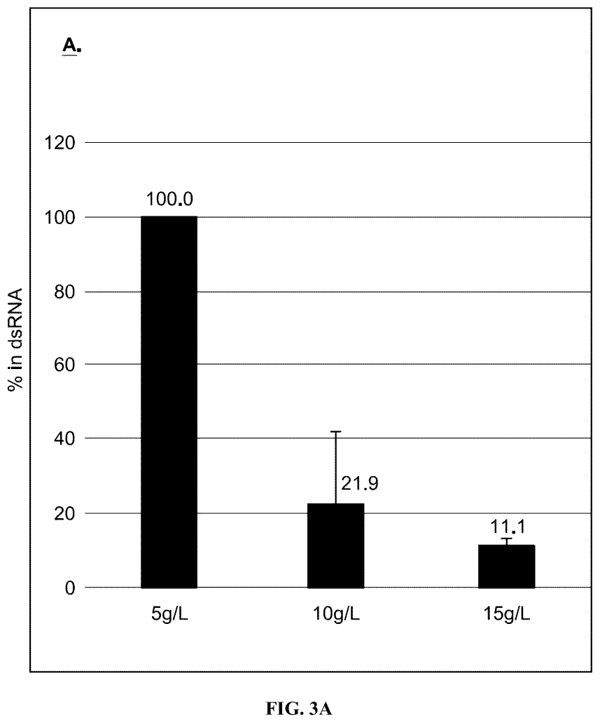

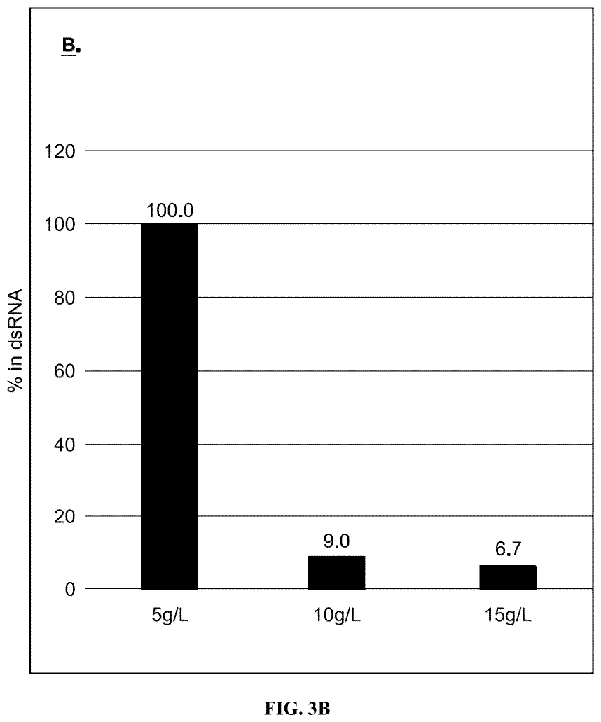

As shown in FIGS. 3a and 3b, both small and large scale 10 g/L and 15 g/L showed 5 to less dsRNA compared to 5 g/L IVT reactions.

(v). In Vitro Transcription Using Organic Solvent Additives Further Reduces dsRNA

The in vitro transcription procedures used in Example 1 were modified by adding a solvent such as ethanol, acetonitrile, isopropanol, or methanol. Additives were added after the WFI and the buffer were heated to 37° C., right before adding the L.DNA. Additives did not affect the time of the IVT reaction. Table 6 below shows the yields and levels of dsRNA when ethanol and acetonitrile were added to the IVT reaction.

TABLE 6

| L.DNA | | | | | | | | | |
|---|---|---|---|---|---|---|---|---|---|
| template | | Mg²⁺ | NTP | Cap | T7 | | Reaction | Yield | |
| with size | L.DNA | conc. | amounts | amount | pol. | Additive | Time | (g/L) | dsRNA |
| 11000 | 0.075 | 30 | 5 mM | 1.5 | 0.025 | — | 50 | 4.2 | 100% |
| nt | mg/mL | mM | each | mM | μg/μL | | min | g/L | ref* |
| 11000 | 0.075 | 30 | 5 mM | 1.5 | 0.025 | 10% | 50 | 4.3 | 19.2% |
| nt | mg/mL | mM | each | mM | μg/μL | ethanol | min | g/L | of ref |
| 11000 | 0.075 | 30 | 5 mM | 1.5 | 0.025 | 5% | 50 | 4.52 | 9.4% |
| nt | mg/mL | mM | each | mM | μg/μL | acetonitrile | min | g/L | of ref |
| 11000 | 0.075 | 50 | 10 mM | 1.5 | 0.025 | — | 120 | 10.2 | 27.9% |
| nt | mg/mL | mM | each | mM | μg/μL | | min | g/L | of ref |
| 11000 | 0.075 | 50 | 10 mM | 1.5 | 0.025 | 7.5% | 120 | 8.6 | 4.4% |
| nt | mg/mL | mM | each | mM | μg/μL | ethanol | min | g/L | of ref |
| 11000 | 0.075 | 50 | 10 mM | 1.5 | 0.025 | 5% | 120 | 8.9 | 2.5% |
| nt | mg/ml | mM | each | mM | μg/μL | acetonitrile | min | g/L | of ref |

*= level of dsRNA of the 11000 nt construct at the 5 g/L condition

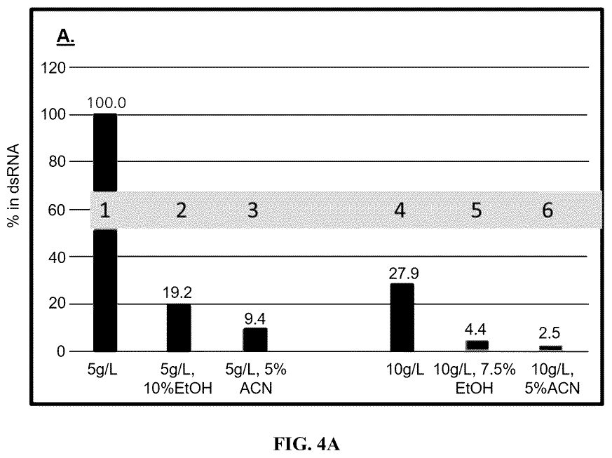

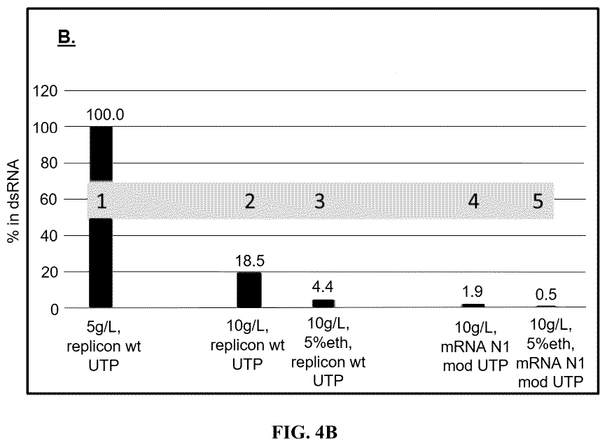

Various amounts of ethanol, acetonitrile, methanol and isopropanol were tested on IVT. Ethanol and acetonitrile when added to the IVT at concentrations between 1-11% v/v results in a significant decrease in dsRNA level. Ethanol and ACN decreased the dsRNA level by 5- to 10-times (quantitated by dot blot as performed in Example 2) independent of the starting level of dsRNA or the target IVT condition (FIG. 4A, compare lanes 2 and 3 to lane 1, and lanes 5 and 6 to lane 4). Acetonitrile showed better dsRNA decrease than ethanol (FIG. 4A, lanes 2 vs 3 and 5 vs 6). The additives showed a decrease in dsRNA whether the mRNA was a large replicon with wt UTP with high dsRNA, (FIG. 4B, lanes 3 vs 2) or smaller mRNA with N¹-methylpseudouridine UTP with low levels of dsRNA (FIG. 4B, lane 5 vs 4).

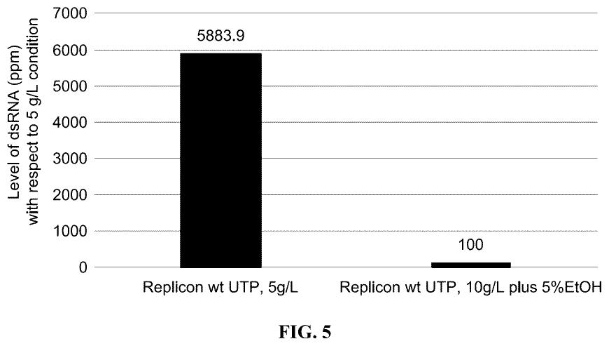

The effect of yield and additives on dsRNA was synergistic as illustrated by FIG. 4A, in lanes 1, 4 to 5 and 6. The results from lane 4 demonstrated the increase in yield effect on dsRNA, and the results from lanes 5 to 6 demonstrated the increase in yield from 5 to 10 g/L plus adding ethanol and acetonitrile. The resultant decrease in dsRNA was between 25-60 times. This was confirmed by Sandwich Elisa assay, which used dsRNA Abs for capture and detection as described in FIG. 5.

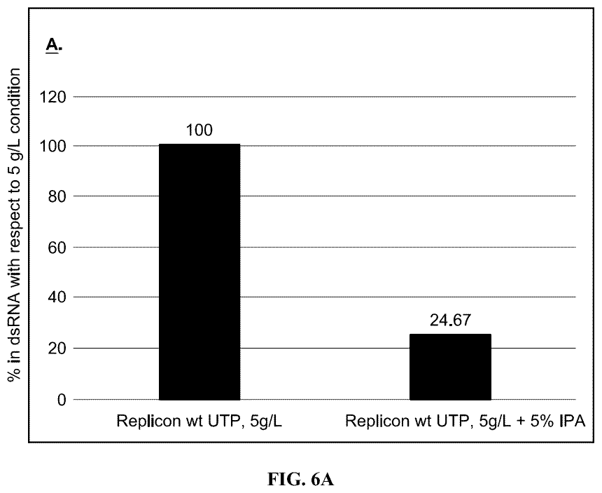

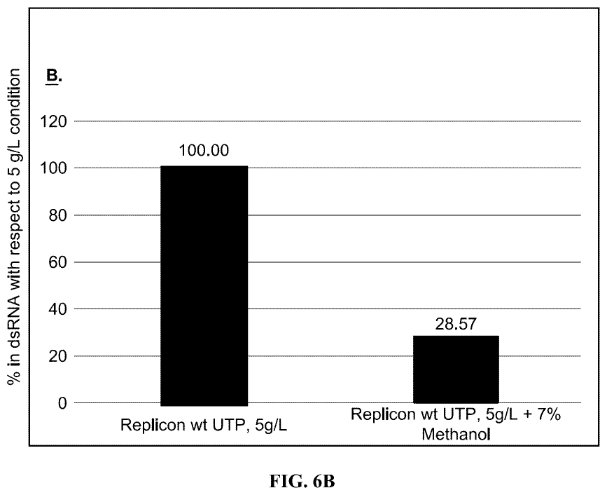

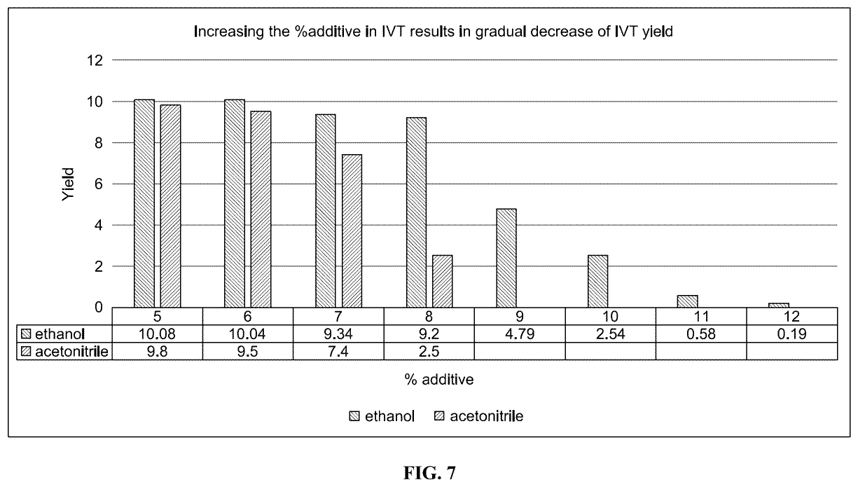

Other additives such as isoproponal and methanol had similar levels on IVT dsRNA decrease, albeit with different levels compared to each other and ethanol and acetonitrile (AcN) (FIG. 6). Higher % v/v of the tested additives resulted in failed IVT yield. For acetonitrile, no considerable yield above 8% v/v was observed, whereas for ethanol, low yields at 10% were observed (FIG. 7).

Thus, the addition of organic solvents such as ethanol, acetonitrile, methanol, and isopropanol provided a surprising decrease in dsRNA without negatively impacting the high yield IVT reactions conditions described in Example 1.

(vi). In Vitro Transcription Using Salt Spiked L. DNA Reduces dsRNA

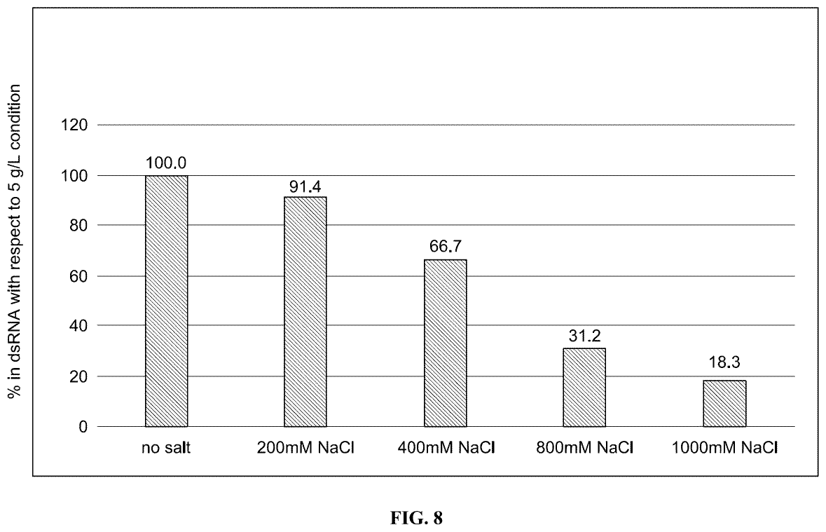

The addition of NaCl to the L.DNA prior to addition to the IVT vessel (called “salt spiking”) was tested as another possible method of lowering the dsRNA of the IVT product. This effect was variable based on the mRNA chemistry, size, and molecule. For the self-replicating mRNAs made with unmodified NTPs, any salt spike level contributed to dsRNA level decrease with significant effects seen with >200 mM NaCl salt spike (FIG. 8). It was observed that salt spikes >1200 mM NaCl will result in IVT yield inhibition.

The combination of high yield, organic additives and DNA salt spike were applied to a large manufacturing scale of in vitro transcription at 4 L with a final 25 g total product. Reaction conditions and a double stranded RNA and product summary are listed in Table 7.

TABLE 7

| L.DNA | | | | | | | | | |
|---|---|---|---|---|---|---|---|---|---|
| template | | | NTP | Cap | T7 | | Reaction | Yield | |
| with size | L.DNA | [Mg²⁺] | amounts | amount | pol. | Additive | Time | (g/L) | dsRNA |
| 11000 | 0.075 | 50 | 10 mM | 1.5 | 0.075 | 5% | 105 | 10.4 | 5.7% |
| nt AU04- | mg/ml | mM | each | mM | μg/μL | ethanol | min | g/L | ref |
| G003^{$} | | | | | | | | | |
| 11000 | 0.075 | 50 | 10 mM | 1.5 | 0.075 | 5% | 105 | 10.0 | 6.2% |
| nt AU03- | mg/ml | mM | each | mM | μg/μL | ethanol | min | g/L | ref |
| G002^{$} | | | | | | | | | |

^{$}lot number of a 25 g mRNA manufacturing scale performed at GMP facility

(vii). Impact of Equimolar versus Non-Equimolar Conditions for In Vitro Transcription on dsRNA Impurity

To thoroughly study the methodology presented herein, studies evaluating the impact of equimolar versus non-equimolar in vitro transcription were conducted in a “head-to-head” investigation. Seven conditions were studied, where the reference methods refer to conditions presented in U.S. Pat. No. 10,653,712.

| | Condition | | | | | | |
|---|---|---|---|---|---|---|---|
| Reagent | 1 | 2 | 3 | 4 | 5 | 6 | 7 |
| Tris-HCl pH 7.5 (mM) | 40.0 | 40.0 | 40.0 | 40.0 | 40.0 | 40.0 | 40.0 |
| Mg²⁺ (mM) | 30.0 | 40.0 | 65.0 | 40.0 | 80.0 | 30.0 | 70.0 |
| DTT (mM) | 10.0 | 10.0 | 10.0 | 10.0 | 10.0 | 10.0 | 10.0 |
| ATP (mM) | 5.0 | 7.5 | 12.0 | 15.0 | 15.0 | 5.0 | 12.0 |
| CTP (mM) | 5.0 | 7.5 | 12.0 | 7.5 | 15.0 | 5.0 | 12.0 |
| GTP (mM) | 5.0 | 7.5 | 12.0 | 30.0 | 30.0 | 10.0 | 24.0 |
| UTP (mM) | 5.0 | 7.5 | 12.0 | 7.5 | 15.0 | 5.0 | 12.0 |
| Capping Reagent (mM) | 1.5 | 1.5 | 1.5 | 1.5 | 1.5 | 1.5 | 1.5 |
| DNA template (mg/mL) | 0.075 | 0.075 | 0.075 | 0.075 | 0.075 | 0.075 | 0.075 |
| RNase Inhibitor (μg/μL) | 0.25 | 0.25 | 0.25 | 0.25 | 0.25 | 0.25 | 0.25 |
| Pyrophosphatase (U/μL) | 0.002 | 0.002 | 0.002 | 0.01 | 0.01 | 0.002 | 0.002 |
| T7 (mg/mL) | 0.0375 | 0.0375 | 0.075 | 0.075 | 0.075 | 0.0375 | 0.075 |

Description of Conditions

Condition 1: Equimolar amounts of NTPs (5 mM) with 1.5 mM of capping reagent, 30 mM of MgOAc, and 0.0375 μg/μL of T7 Polymerase.

Condition 2: (Reference condition “Equimolar”) Equimolar amounts of NTPs (7.5 mM) with 1.5 mM of capping reagent, 40 mM of MgOAc, and 0.0375 μg/μL of T7 Polymerase.

Condition 3: Equimolar amounts of NTPs (12 mM) with 1.5 mM of capping reagent, 65 mM of MgOAc, 0.0750 μg/μL of T7 Polymerase, and no ethanol.

Condition 4: (Reference condition “Alpha”) 30 mM GTP, 15 mM ATP, 7.5 mM for CTP and UTP, 1.5 mM capping reagent, 40 mM, MgOAc, 0.0750 μg/μL of T7 Polymerase, and 5-times iPP amount at 0.01 U/μL.

Condition 5: (Reference Condition “Alpha” modified with present experimental methodology) 30 mM GTP, 15 mM of ATP, CTP, and UTP, 1.5 mM of capping reagent, 80 mM of MgOAc, 0.0750 μg/μL of T7 Polymerase, and 5-times iPP amount at 0.01 U/μL.

Condition 6: (Condition 5 modified) non-equimolar amounts using 10 mM GTP and 5 mM of ATP, CTP, and UTP, 1.5 mM of capping reagent, 30 mM of MgOAc, and 0.0375 μg/μL of T7 Polymerase.

Condition 7: (Reference Condition “Alpha” modified with present experimental methodology) 24 mM GTP, 12 mM of ATP, CTP, UTP, 1.5 mM of capping reagent, 70 mM of MgOAc, 0.0750 μg/μL of T7 Polymerase, and 5-times iPP amount at 0.01 U/μL.

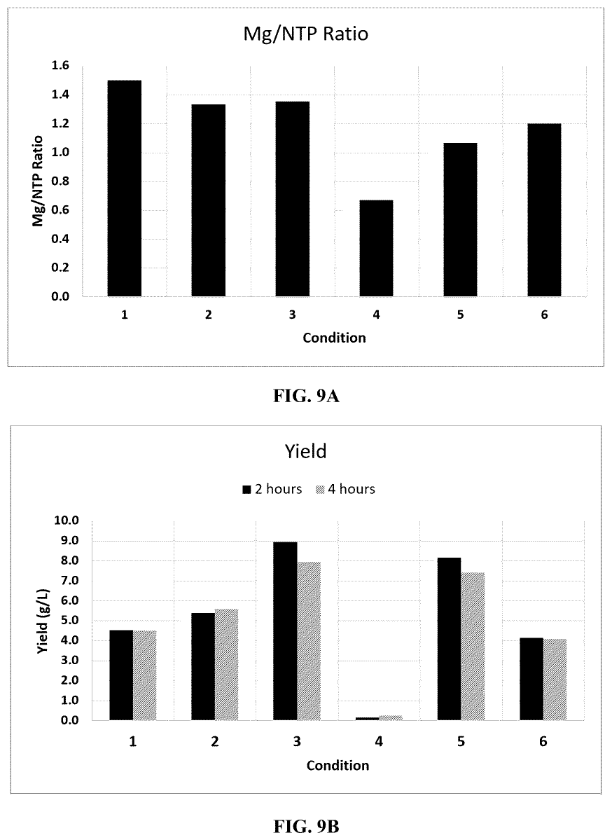

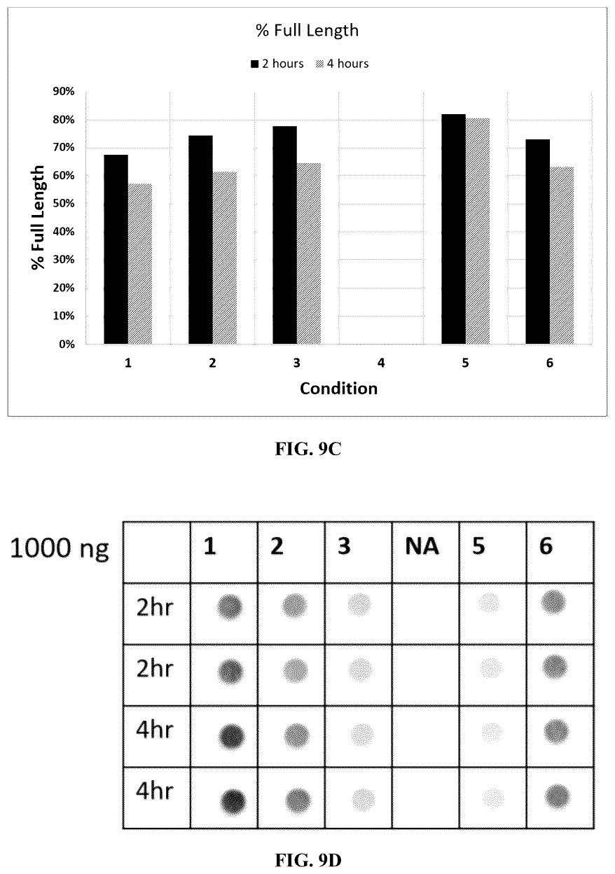

Turning to the results of conducting in vitro transcription reactions with the above various conditions, FIGS. 9A-9C illustrate several results. FIG. 9C, which illustrates the percent of full length transcripts that resulted from IVT, shows that the reaction completely fails when there is not enough Mg²⁺ present such as Condition 4. The results of Condition 5 in FIG. 9C suggests that that there is enough Mg²⁺ present, but not in excess, thereby a slight decrease in purity. Additionally, there is a direct correlation between the Mg/NTP ratio (FIG. 9A) versus yield (FIG. 9B) and indirect correlation between Mg/NTP ratio (FIG. 9A) versus % Full length (FIG. 9C) (data plot not shown). Condition 4 is a comparative condition labeled “alpha” according to Table 1 of U.S. Pat. No. 10,653,712. It is also clear that equimolar use of NTPs does not necessarily increase the yield. This is most apparent in a comparison between condition 1 versus condition 6 or condition 3 versus condition 5 in FIG. 9B. Lastly, there is no significant increase in yield between running the reaction for 2 hours and 4 hours.

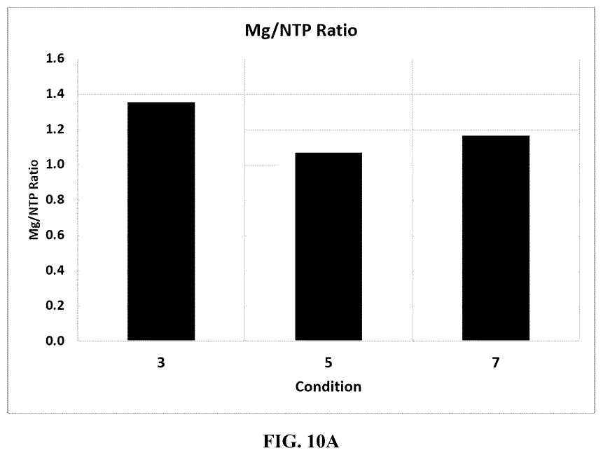

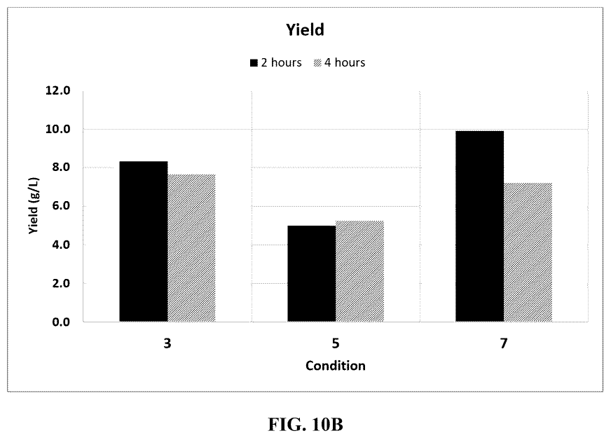

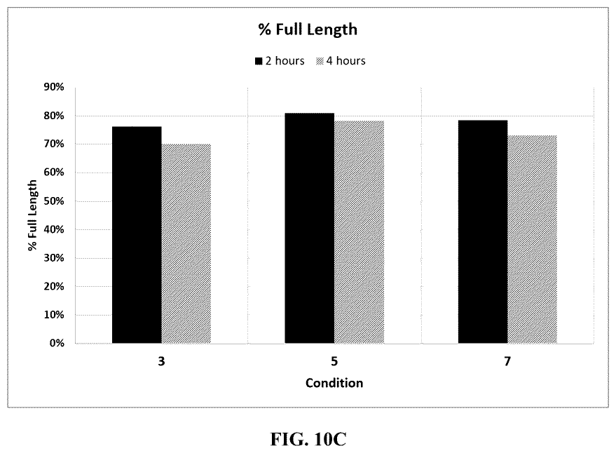

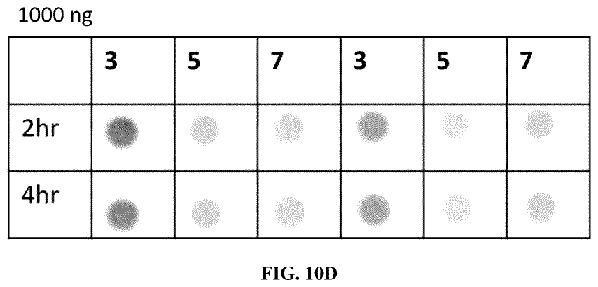

The preceding experiment was repeated with conditions 3 and 5, and with a new experimental condition 7 to provide a more direct analysis of the experimental conditions versus the reference conditions. In the repeated experiments, a direct correlation between Mg²⁺/NTP ratio (FIG. 10A) and yield (FIG. 10B) was observed again as well as an indirect correlation between Mg²⁺/NTP ratio (FIG. 10A) and % Full-length transcript (FIG. 10C) (data plot not shown). Turning to FIG. 10D, Condition 7 (both runs) has lower dsRNA than Condition 3.

In a further experiment, conditions 3 and 7 were scaled-up to 12 g/L target reactions, where salt-spiking and the presence of ethanol were varied for each. Description of conditions:

Condition 3: Equimolar amounts of NTPs (12 mM) with 1.5 mM of capping reagent, 65 mM of MgOAc, 0.0750 μg/μL of T7 Polymerase, iPP amount at 0.002 U/μL and no ethanol.

Condition 3 +Salt Spiking: Equimolar amounts of NTPs (12 mM) with 1.5 mM of capping reagent, 65 mM of MgOAc, 0.0750 μg/μL of T7 Polymerase, iPP amount at 0.002 U/μL salt spike (82 mM), and no ethanol.

Condition 3 +Salt Spiking +Ethanol: Equimolar amounts of NTPs (12 mM) with 1.5 mM of capping reagent, 65 mM of MgOAc, 0.0750 μg/μL of T7 Polymerase, iPP amount at 0.002 U/μL salt spike (82 mM), and 3% v/v ethanol.

Condition 7: (Reference Condition “Alpha” modified with present experimental methodology) 24 mM GTP, 12 mM of ATP, CTP, UTP, 1.5 mM of capping reagent, 70 mM of MgOAc, 0.0750 μg/μL of T7 Polymerase, and iPP amount at 0.002 U/μL.

Condition 7 +Salt+Ethanol: (Reference Condition “Alpha” modified with present experimental methodology) 24 mM GTP, 12 mM of ATP, CTP, UTP, 1.5 mM of capping reagent, 70 mM of MgOAc, 0.0750 μg/μL of T7 Polymerase, iPP amount at 0.002 U/μL salt spike (82 mM), and 3% v/v ethanol.

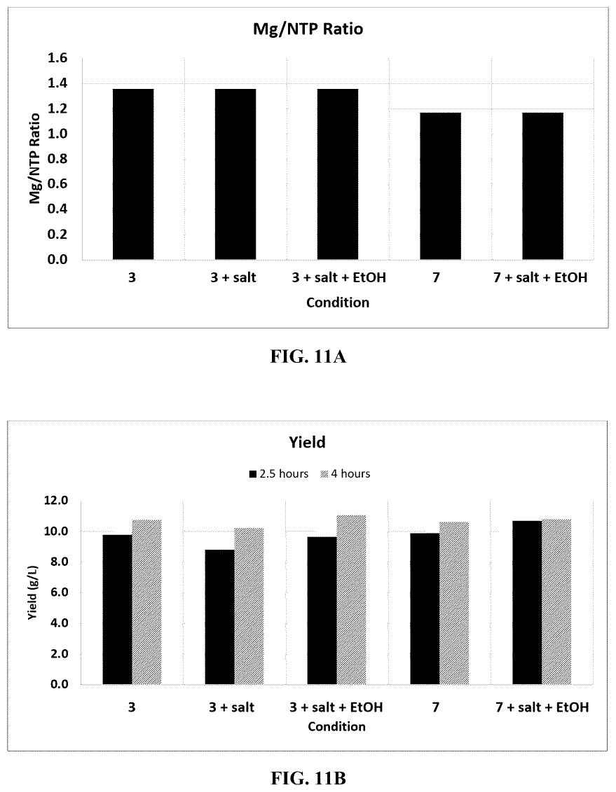

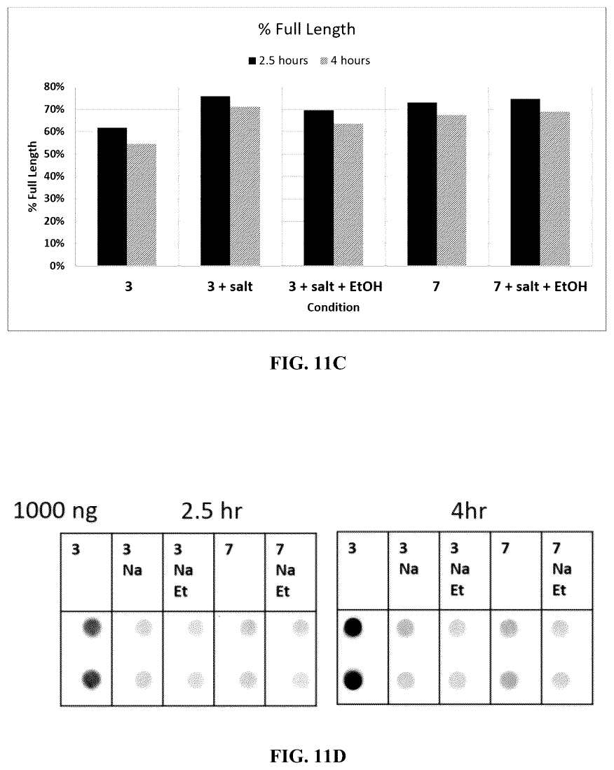

The results from the large-scale IVT reactions show that yield is not impacted by varying the molar amounts of the NTPs (equimolar versus non-equimolar) (see, e.g., FIGS. 11A-11C.) Additionally, FIG. 11D illustrates that comparative condition 7 has lower dsRNA amount, however that result is no longer observed with the presence of salt-spiking and ethanol (see, e.g., last column).

In conclusion, when the NTP to Mg²⁺ concentration was greater than 1 (i.e., not enough Mg²⁺), the reaction failed. Contrary to the reference methodology, non-equimolar amounts of NTPs does not increase the yield, but does appear to lower the presence of dsRNA. However, once NaCl spiking into the L.DNA and an amount of ethanol is introduced, there is no additional benefit from using non-equimolar amounts of NTPs.

(viii). Impact of Denaturant Versus Solvent for In Vitro Transcription on dsRNA Impurity

The present application demonstrates the correlation between mRNA yield and levels of dsRNA. However, elsewhere it has been reported denaturants at certain ranges enhance the processive transcriptional activity of T7 RNA polymerase (see, e.g., Das, M. and Dasguta D. FEBS Letters 1998, 427, 337-340.) A further study was conducted to determine the impact of denaturant (guanidine hydrochloride) on dsRNA impurity level and compare to that of a solvent (e.g., ethanol) to thoroughly study the methodology presented herein.

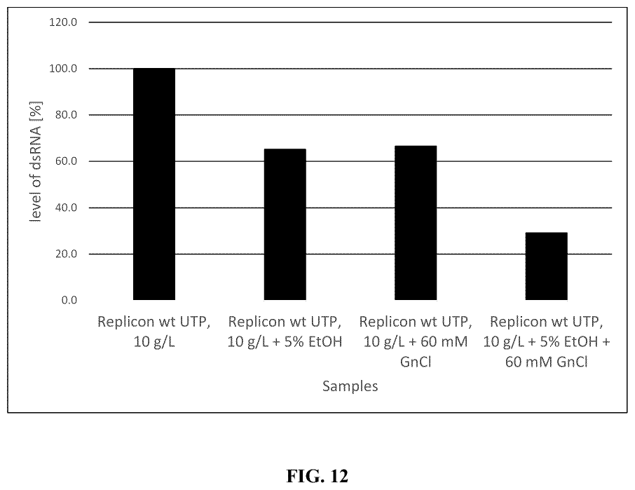

FIG. 12 illustrates the % dsRNA of the mRNA sample relative to the control, which did not contain any denaturant or solvent but was made using the high-yield IVT condition. Adding 5% ethanol or 60 mM guanidine hydrochloride into the IVT mixture resulted in about 33 to 35% reduction in dsRNA: 65.2% and 66.6% relative levels, respectively. When both 5% ethanol and 60 mM guanidine hydrochloride were added to the IVT reaction mixture, there was a further decrease (about 70%) in dsRNA: 29.2% relative dsRNA level. No significant difference in mRNA yield between these samples was observed (data not shown.)

The results demonstrate that the dsRNA reduction using a denaturant, such as guanidine hydrochloride, gives a comparable reduction to a solvent. Furthermore, the results show that there was an additional benefit when the denaturant was used in combination with any of the embodiments described herein, such as high yield condition and solvents as additives.

The examples and embodiments described herein are for illustrative purposes only and in some embodiments, various modifications or changes are to be included within the purview of disclosure and scope of the appended claims.

1. A method of producing a transcribed RNA product comprising:
a. reacting a transcription reaction mixture comprising a buffer solution comprising Mg²⁺, linear DNA (L.DNA) template, ribonucleoside tri-phosphate (rNTPs), optionally an RNA capping reagent, and RNA polymerase; wherein a molar concentration of Mg²⁺ is 2 to 15 mM above the total molar concentration of all rNTPs plus the optional RNA capping reagent; and a RNA polymerase/L. DNA template mass ratio is between 0.25 and 3;
b. stopping the transcription reaction by digesting the L.DNA template with deoxyribonuclease (DNase) or quenching the enzyme with ethylenediaminetratracetic acid (EDTA);
wherein a yield of about 1 g to about 25 g of single stranded transcribed RNA per liter of liquid of the transcription reaction prior to the addition of DNase in step (b) is produced.

2. The method of claim 1, wherein the L.DNA template is in a solution comprising 50 mM to 1200 mM NaCl to produce a salt-spiked L.DNA template prior to introduction to the transcription reaction mixture.

3. The method of claim 1, wherein the transcription reaction mixture of step (a) further comprises one or more of the group consisting of RNase inhibitor and inorganic pyrophosphatase.

4. (canceled)

5. The method of claim 1, wherein the temperature during step a) and b) is in a range from about 30° C. to about 40° C.

6. The method of claim 1, wherein the L.DNA template is from about 0.01 mg/mL to about 0.3 mg/mL in the transcription reaction mixture.

7. The method of claim 1, wherein the RNA polymerase is T7 polymerase.

8. (canceled)

9. The method of claim 1, wherein the transcription reaction mixture of step (a) is allowed to react for at least 20 minutes prior to step (b) stopping the transcription reaction.

10. The method of claim 1, wherein the transcription mixture of step (a) further comprises a transcription initiating RNA capping reagent.

11. The method of claim 1, wherein the molar concentration of Mg²⁺ is 5 to 15 mM above the total molar concentration of all rNTPS plus the molar concentration of any optional RNA capping reagent.

12. The method of claim 1, wherein the RNA polymerase is 0.0125 to 0.15 μg/μL T7 polymerase.

13. (canceled)

14. The method of claim 1, wherein the amount of single stranded transcribed RNA is measured after purification of the transcription mixture after step (b) via a silica column.

15. The method of claim 1, wherein the amount of single stranded transcribed RNA is measured after purification of the transcription mixture after step (b) via affinity column chromatography.

16. The method of claim 15, wherein purification of the transcription mixture after step (b) via a silica column is performed before purification via affinity column chromatography.

17. The method of claim 1, wherein the RNA transcription mixture of step (a) further comprises one or more solvents selected from the group consisting of EtOH in a concentration of 1 to 10% v/v, i-PrOH in a concentration of 1 to 10% v/v, MeOH in a concentration of 1 to 10% v/v, and acetonitrile of a concentration of 1 to 8% v/v, with the proviso that the total concentration of EtOH, i-PrOH, MeOH and acetonitrile does not exceed 10% v/v of the RNA transcription mixture of step (a).

18.-21. (canceled)

22. The method of claim 1, wherein the L.DNA template comprises an open reading frame that encodes a vaccine antigen, enzyme, antibody, receptor, tRNA, and/ or a protein.

23. The method of claim 1, wherein the total concentration of rNTPs is at least 8 mM.

24. The method claim 1, wherein a yield of greater than about 5 g/ L of RNA transcript results in a reduced amount of dsRNA as compared to an otherwise identical transcription reaction wherein the yield is less than about 5 g/L.

25. A method of producing a transcribed RNA product comprising:
a. loading a reactor vessel with a buffer solution comprising Mg²⁺ and nuclease free water;
b. adding a solution comprising L.DNA template spiked with 200-1000 mM NaCl;
c. adding rNTPs (define as wild-type and analogs) and optionally RNA cap (define in specification);
d. adding RNA polymerase and mixing the resulting transcription solution wherein the RNA polymerase/DNA template mass ratio is between 0.25 and 3; and,
e. adding DNase to the transcription solution;
wherein the molar concentration of Mg²⁺ is 2 to 15 mM above the total molar concentration of all rNTPs plus the optional RNA cap after steps (a)-(b) are completed, and wherein 1 to 25 g of single stranded transcribed RNA/ L of liquid of the transcription reaction prior to the addition of DNase in step (b) is produced.

26. A method of producing a transcribed RNA product with reduced dsRNA comprising:
a. reacting a transcription reaction mixture comprising a buffer solution comprising Mg²⁺, L.DNA template previously spiked with 50 to 1200 mM NaCl, rNTPs, optionally RNA cap, RNA polymerase wherein the RNA polymerase/L. DNA template mass ratio is between 0.25 and 3, and one or more solvents selected from the group consisting of EtOH in a concentration of 1 to 10% v/v, i-PrOH in a concentration of 1 to 10% v/v, MeOH in a concentration of 1 to 10% v/v, and acetonitrile of a concentration of 1 to 8% v/v, with the proviso that the total concentration of EtOH, i-PrOH, MeOH and acetonitrile does not exceed 10+/−1% v/v of the RNA transcription mixture; and,
b. adding DNase to the transcription reaction mixture;
wherein the molar concentration of Mg²⁺ in the transcription reaction mixture is 2 to 15 mM above the total molar concentration of all rNTPs plus the optional RNA cap.

27. The method of claim 25, wherein a yield of greater than about 5 g/L of RNA transcript results in a reduced amount of dsRNA as compared to an otherwise identical transcription reaction wherein the yield is less than about 5 g/L.28.

28. (canceled)
